(19) United States
(12) Patent Application Publication
(10) Pub. No.: US 2003/0095988 A1
(43) Pub. Date: May 22, 2003

(54) Method of inducing autovaccination against HIV infection using structured treatment interruptions

(76) Inventors: Julianna Lisziewicz, Bethesda, MD (US); Franco Lori, Bethesda, MD (US); Jianqing Xu, Silver Spring, MD (US); Georg Stephen Varga, Washington, DC (US)

Correspondence Address: The Law Offices of Valerie E Looper, 11726 Lightfall Court, Columbia, MD, 21044, US

(21) Appl. No.: 09/493,769

(22) Filed: Jan. 28, 2000

(51) Int. Cl. C12Q 1/68; A61K 31/70; A01N 43/04; A61K 39/21; C07K 2/00; C07K 4/00; C07K 5/00; C07K 7/00; C07K 14/00; C07K 16/00; C07K 17/00; A61K 38/00

(52) U.S. Cl. 424/208.1; 435/6; 424/207.1; 514/44; 530/300

(57) ABSTRACT

Structured Treatment Interruptions of drug therapy for Human Immunodeficiency Virus infection can be used to enhance HIV-specific immune responses, thereby allowing the individual to control viral replication after interrupting the drug treatment. Immunoregulatory adjuvants can further increase these immune responses. A diagnostic method for the immune status of the patient that controls HIV includes measurements of viral load and production of both IFN-gamma and IL-10.

### FIELD OF THE INVENTION

The present invention relates to the use of autovaccination techniques for the treatment of disease in mammals. Autovaccination is the use of a pathogen already present in the body to induce immune system responses. The result of autovaccination is control of the pathogen by the immune system of the infected individual in the absence of drug treatment. The inventors have demonstrated that, under the right conditions, autovaccination can be induced by structured interruptions of drug treatment for viral infections. They have also identified the desired responses in the case of HIV infection as antigen-specific T cell responses, and shown that this technique can be used to help infected individuals achieve immune control of HIV infection. Further, various adjunct treatments to enhance the effect of the structured treatment interruptions are described, as well as a diagnostic assay to predict the success of the autovaccination technique for a given infected individual.

### BACKGROUND OF THE INVENTION

This is the year the number of deaths from HIV infection will reach the number of deliberate civilian murders by the Nazi regime of World War II (16 million plus). With another 30 million infected worldwide, and with the unimpeded spread of the infection in some areas of the world, the expected number of deaths assuming effective treatment campaigns are implemented immediately will exceed all casualties of both world wars. As a result, discussion of the AIDS epidemic by the United Nations Security Council (Reuters Health, Jan. 11, 2000) has turned to the strategic implications of the enormous loss of life caused by this infection.

The problem of the AIDS pandemic is so severe that some activists have begun to ignore the reality of twenty-plus years of heavy research and development investment in fighting this disease. They choose to ascribe the lack of a vaccine to lack of interest (Washington Post, Jan. 18, 2000, p. A17). In fact, all of the classical methods of vaccination have been explored, and found inadequate to the challenges presented by HIV and researchers are actively pursuing new information and new theories (Journal of Virology/Medscape Wire Jan. 13, 2000).

Some tools and partial answers do exist for handling this disease. Highly Active Antiretroviral Therapy (HAART) has been shown to suppress viral replication in many infected individuals. Many forms of HAART therapy rely on powerful drug combinations, however, that can present toxicity problems. With most HAART regimens, cessation of drug use results in an immediate resumption of viral replication, or viral rebound, in the individual. Many HAART regimes lose their effectiveness over time, so that the number of viral particles found in an individual's fluids or tissues (that is, the patient's viral load in a given fluid or tissue) eventually begins to rise, or rebound, despite continued drug use. Further, many HAART regimens are complicated enough that many individuals have difficulty complying, (J. Acquir Immune Defic Syndr 1999; 22:498-502, abstract at Reuters Health Jan. 13, 2000), so that some patients never respond properly to the medication (J Acquir Immune Defic Syndr 1999;22:358-363, abstract at Reuters Medical News Jan. 11, 2000) and failure to comply is associated with both viral rebound and the development of drug-resistant viral strains, although the two results appear to be separable (JAMA 2000;283:205-211,229-234,250-251, abstract at Medscape Wire Jan. 11, 2000). When resistant strains of HIV, also known as escape mutants, are present, they may demonstrate an even greater replicative capacity than the wild-type strain (AIDS 199;1 3:2349-2359, abstract at Reuters Medical News Dec. 30, 1999). Finally, although some HAART regimens have been shown to suppress viral reproduction, some researchers have suggested that delaying the start of such therapy may have no clinical effect (AIDS 1999; 13:2547-2554, abstract at Reuters Medical News Jan. 10, 2000).

While HAART regimens are only a partial answer to the challenges posed by this epidemic, they have had a dramatic, positive impact on the health of people with HIV infections. They are the most effective tools currently available against HIV, and their utility might be greatly expanded by exploring various different ways to apply them. With a better understanding of the functions of the immune system and different methods of using the drugs, more effective treatments, lower toxicities and lower costs may be achieved. Some clues do exist that point to possibilities for new modes of therapy.

A few individuals have been found who appear to have a natural ability to control the virus. These individuals have been found, among other things, to have strong HIV-specific, T cell mediated, immune responses. Infected individuals have been shown to have such responses, but lose them over time if untreated. Similarly, individuals on some HAART regimens have been shown to have such responses but lose them over time, despite what appears to be complete suppression of the virus by the HAART regimen.

The inventors have reported their discovery that a combination of hydroxyurea, a reverse transcriptase inhibitor, and a protease inhibitor can be used to inhibit HIV in human beings, with greatly improved results in that viral rebound may be delayed for at least three to eight weeks or more. See U.S. Pat. No. 5,977,086, issued Nov. 2, 1999. These results indicate that the triple combination which includes hydroxyurea may be used for the treatment of HIV infection. The inventors have found that the double combination of hydroxyurea and a reverse transcriptase inhibitor can also be used, without the addition of a protease inhibitor, for long-term treatment of HIV infections, without provoking viral rebound, and also that use of an immune system stimulant such as a vaccination or a cytokine known to activate quiescent cells may be useful. See U.S. Ser. No. 09/048,886, Method of Inhibiting Human Immunodeficiency Viruses using Hydroxyurea and a Reverse Transcriptase Inhibitor, and U.S. Ser. No. 09/048,576 “Method of Rendering a Human Immunodeficiency Virus Population Replication Incompetent in vivo both filed Mar. 26, 1998 and incorporated by reference herein as if set forth in full. The inventors confirmed that hydroxyurea-containing HAART regimens can be used in an autologous vaccination technique. That is, the virus that has already infected the individual can be manipulated to act as a vaccine that generates an HIV-specific immune response. This immune response allows the individual's body to control the rate of HIV replication after drug therapy is withdrawn. See U.S. Ser. No. 09/243,753, filed Feb. 3, 1999. Each of the above-described patents and applications by the inventors is incorporated herein as if set forth in full.

There is currently some interest in exploring autologous vaccination techniques, or structured treatment interruption, in an effort to teach the immune systems of infected patients to handle HIV infection (Science, Vol. 286, Nov. 19, 1999, www.science.org), although the inventors' ideas have been characterized as “heresy” (New Scientist, Mar. 27, 1999), and the FDA has taken umbrage at a drug manufacturer for reporting good results obtained using hydroxyurea, a cancer drug which is not yet approved for use in the United States in connection with HIV. Nonetheless, various methods of boosting the immune system are being considered in many quarters. (For a good review, see “Boosting Immune Function The Next Step in HIV Therapy?” Dec. 8, 1999 www.hivandhepatitis.com/hivtreatmentlive/html). The pool of good candidates for HIV treatment continues to expand, and includes immune therapy agents such as interleukin-2 (the AIDS reader 9(8):519-529, 1999 and Effect of Interleukin-2 on the pool of latently infected, resting CD4+ T cells in HIV-1-infected patients receiving highly active anti-retroviral therapy, Chun, et al., Nature Medicine Vol. 5 Number 6, pp 651-655, June 1999).

The inventors have now discovered that drug treatments, including but not limited to hydroxyurea-containing HAART, can be used to increase the competence of a patient's immune system through structured treatment interruptions, various adjunct therapies that can be used to heighten the effect, and a group of assays to indicate whether such competence has been achieved.

### BRIEF DESCRIPTION OF THE DRAWINGS

FIG. 1 is a schematic drawing of the dose-dependent response of an immune system to an antigen.

FIG. 2 is a schematic drawing of the response of the human immune system to HIV viral load in the plasma, comparing various populations treated with various drug regimens, including continuous therapies and STI.

FIG. 3 shows viral load over time for three patients during STI therapy.

FIG. 4 shows the viral load data for 29 monkeys infected with SIV and treated with continuous HAART and STI various drug combinations.

FIG. 5 shows the viral load data for 3 groups of monkeys infected with SIV. The control group was untreated, and the other two groups were treated with either HAART or one of several intermittent therapies based on ddl+PMPA+HU.

FIG. 6 shows the CD4 counts for 29 monkeys infected with SIV at initiation of therapy, during therapy, and 41 days after cessation of therapy.

FIG. 7 shows viral load data (FIG. 7A), together with CD4 counts and CD8 counts(FIG. 7B) for the Washington patient during STI treatment. A schematic diagram at the top represents the STI periods. Treatment periods are labeled with numbers from 1 to 5 (white background); interruption periods are labeled with letters from A to E (grey background).

FIG. 8 shows results of a flow cytometric assay for HIV-specific T cell responses (VIRs) by IFN-gamma production of different subsets of lymphocytes. Row a shows HIV-specific CD3+, CD8+ and CD4+ T cell responses detected after stimulation with HIV antigen in one representative seronegative individuals (negative controls, NC). B, C and D) Immune reponses of one seropositive individual (positive control, PC). This individual controlled HIV in the absence of drug treatment. B) Background of the CD3+, CD8+ and CD4+ T cell responses detected in the absence of antigenic stimulation. C) Non-specific CD3+, CD8+ and CD4+ T cell responses detected after stimulation with control antigen (lysosyme). D) HIV-specific CD3+, CD8+ and CD4+ T cells detected after stimulation with HIV antigen.

FIG. 9 shows HIV-specific T cell responses during interruption periods for the Washington patient treated with STI. The first column represents CD3+, CD8+ and CD4+ T cell responses after stimulation with a control antigen (lysozyme). Columns A, C, and E represent CD3+, CD8+ and CD4+ T cell responses after stimulation with HIV antigen during interruptions A, C and E (see FIG. 1). HIV-specific IFN-gamma responses are shown in the CD3+ T cell population (row a), in the CD8+ subpopulation of T cells (row b), in CD4+ subpopulation of T cells (row c)

FIG. 10 shows CTL activity of the Washington patient treated with STI during the last treatment interruption. HIV antigen-specific lysis (closed diamond) and control lysis (open square) were assayed using different effector:target ratios (E:T).

FIG. 11 shows HIV-specific memory cell responses during STI. Row A shows IFN-gamma responses in three groups of T cells (CD45RA+, CD45RO+ and CD45RA,CD45RO+ T cells) after HIV-specific stimulation of PBMC derived from the PC patient. For this assay CD45RO antibody was used. Row B shows HIV-specific CD3+,T cell responses from CD45RO+ T cells after stimulation with HIV antigen in one representative seronegative individual (left panel). Memory T cell responses against a control with no antigen and with a control antigen, lysozyme (middle two panels) and against HIV (right panel) from one seropositive individual (PC) who controlled HIV in the absence of drug treatment. Row c shows HIV-specific memory T cell responses of the Washington patients during STI. Non-specific IFN-gamma production in CD45RO+,CD3+ T cells after stimulation with a control antigen (left panel). HIV-specific IFN-gamma responses in CD45RO+,CD3+ T cells during the interruptions of A, C and E (see FIG. 1).

FIG. 12 shows IFN-gamma response during in vitro activation with HIV antigen in CD3+ cells and CD3− cells before and during one treatment phase of STI therapy, in association with the viral load of the patient.

FIG. 13 shows the percent of IL-10 producing cells from four different classes. The assay was done 9 days before the 5^{th} therapy cycle began for the Washington patient (see FIG. 7). HIV antigen decreased IL-10 production and IL-10 antibody restored the effect of HIV. The effect was visible only in the T cell (CD3+) population, especially in the CD8+ subgroup.

FIG. 14A shows spontaneous IFN-gamma production and 14B shows HIV-specific IFN-gamma production in the CD3+ T cell and CD3− cell populations before and after suppression of the viral load.

FIG. 15 shows stimulation of HIV-specific TH1 responses by IL-2 and IL-10 neutralizing antibodies as measured as percentage of IFN-gamma producing cells.

### DETAILED DESCRIPTION OF THE INVENTION

Autovaccination

Autovaccination is a technique for raising an immune response in a given body using a pathogen present in the body. The technique works in the same way as a normal vaccination, except that normal vaccination provides antigens prepared in a laboratory. As is the case with vaccination, the goal of autovaccination is the immune control of the pathogen. That is, control of the pathogen by the body in the absence of drug treatment. The inventors have previously shown that autovaccination against HIV can be induced by long-term administration of a specific drug regimen that controls the total amount of virus in the body within detectable levels in the presence of ongoing viral replication, and by structured treatment interruptions (STI) using the same regimen. See U.S. Ser. No. 09/243,753, above. Here we describe a general method of STI that can be used with certain classes of pathogens, together with the type of immune response that must be induced to achieve immune control of HIV replication, a diagnostic assay to predict the success of the autovaccination, and adjunct therapies to enhance the effect.

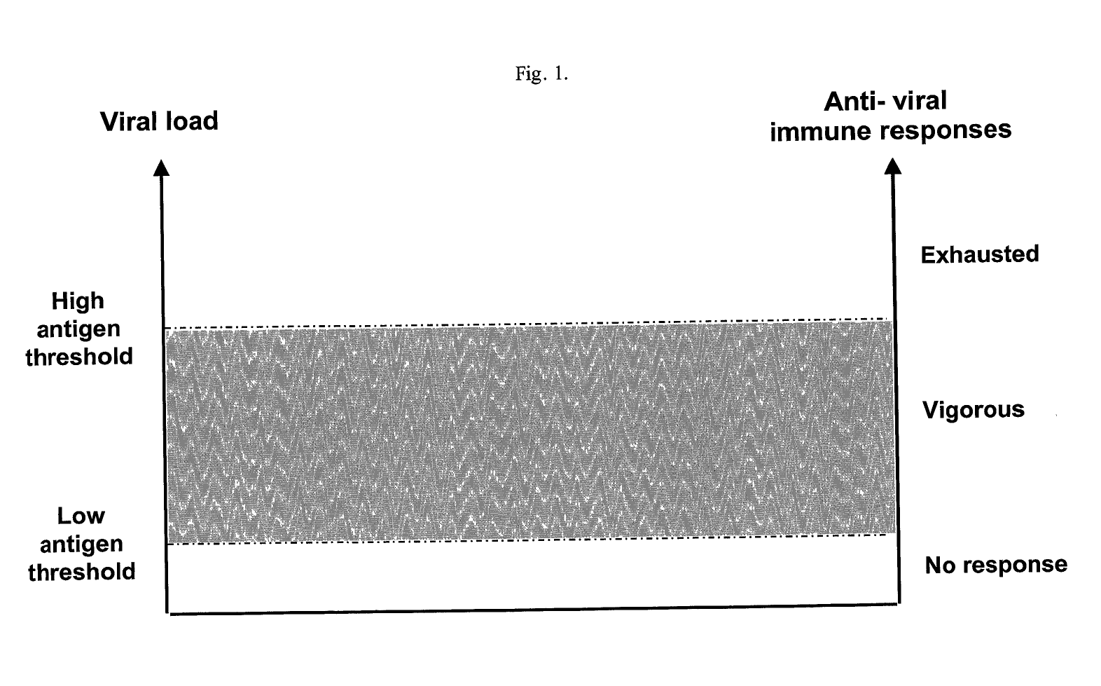

The human immune system has a variety of ways to respond to challenge by the many chemicals and pathogens, such as viruses, bacteria, parasites and malfunctioning cells, which may cause it harm. These materials are recognized by the immune system as foreign antigens. The immune system reacts to antigens in a dose dependent manner. Too little antigen will fail to induce an immune response. Too much of an antigen will overwhelm and exhaust the immune system. In order to induce an immune response that is able to keep the pathogen under control, the antigen must be present in an optimal dose, that is, a dose between the low antigen threshold and high antigen threshold. See FIG. 1. In the present invention, drug treatments can be used to control the autologous virus in a manner to deliver an optimal dose of antigen in two ways: a strong therapy, such as HAART, that is a therapy that can drive the viral load below the level of detectability within 2-4 3 weeks, can be administered in an intermittent fashion. In that case, the antigen is delivered for a short period of time, that is, rebound of the autologous virus is limited by renewed drug treatment. In that case, the optimal dose is reflected by a viral load of 10,000-100,000 copies/ml plasma. A therapy that does not drive down the viral load so quickly can be used, provided the virus can be controlled for longer periods of time within the detectable range. An example of such a therapy is the combination of hydroxyurea and ddl, optionally with d4T. In that case, the optimal dose of antigen is delivered over a longer period of time, perhaps up to a year or more, which is reflected in a lower range for the viral load in the plasma, preferably about 200-500 to 10,000 copies/ml. After the patient's viral load drops below the 200-500 copies/ml range, however, the patient can be placed on an intermittent schedule.

One class of immune responses, T cell mediated immune responses, is known to be present when viruses, other intracellular parasites (e.g. malaria) and tumors are controlled by an animal's immune system. T cell mediated immune responses kill infected cells or tumor cells. Therefore, this method (structured treatment interruptions (STI) to achieve autovaccination) is applicable to all the viruses, other intracellular parasites and tumors, provided an at least partially effective drug therapy exists.

In the case of viruses, a successful antiretroviral drug therapy inhibits virus replication and decreases the number of copies of the virus, or viral load, by 99.9% or more in a patient's fluids or tissues. In the case of HIV, inhibition of viral replication results in a partial reconstitution of the immune system. For example, many HAART regimens can inhibit HIV replication to the point that the viral load in the patient's plasma is undetectable (less than 500 copies per ml, 400 copies per ml, or 200 copies per ml, depending on the test). At the same time, the patient's immune responses against other pathogens also recover. However, the patient's immune responses against HIV do not recover, but continue to decline over time. Some regimens, particularly the hydroxyurea-didanosine regimens that maintain a low but detectable viral load in the patient's plasma, do not necessarily demonstrate as such a decline [Lori et al. Aids Research and Human Retroviruses 1999 and previous application]. ]Here we describe a new technology that can be used to increase the ability of a body's immune system to control a pathogen after interruption of therapy. We present various pieces of evidence that STI is as effective to control HIV replication as continuous therapy and, more importantly, can induce immune responses, identified as HIV-specific T cell responses, that can control HIV after interruption of STI treatment.

An example of another viral infection amenable to the present invention is Hepatitis B (HBV). This virus can be successfully treated with antiretrovial drugs such as 3TC, however, like HIV, HBV can form escape mutants that result in a strain that resists drug treatment. We suggest that structured treatment interruptions can be used to enhance the patient's HBV-specific T cell responses. In this case, adding hydroxyurea to the regimen is preferred. Short periods (preferably1-6 weeks) of drug therapy can be followed by treatment interruptions. HBV-specific T cell mediated immune responses (measured with the method described here) will determine how long HBV can be controlled in the absence of drugs, similarly to the situation we describe here with HIV and SIV.

At the present time, lymphomas (Hodgkin and non-Hodgkin), small cell carcinoma of the lung, testicular cancers, coriocarcinomas have successful therapies meaning that complete remission can be achieved. All other cancers have lower percentage of remissions after therapy. All of these therapies can be given for short periods of time until the tumor-specific T cell responses develop (measured with the method described here). These responses will determine how long the patient will control the tumor, similar to the case of HIV and SIV as described here. At the first signs of relapse, short therapy must be given again and again in order to achieve a long-term remission, as in the case of the Berlin and Washington patients, described in Examples 1 and 4, below.

Autovaccination to Control HIV

In the case of viral infection, autovaccination is the technique of using the patient's own, autologous virus to induce virus-specific immune responses. The virus-specific immune responses induce control of the replication rate of the virus. Much like vaccination, autovaccination requires the presence of viral antigens to induce immune responses. While vaccination may rely on nonliving subunits of a virus, various chemicals that mimic parts of the virus, killed virus or weakened viral particles, autovaccination relies on manipulation of the size of the autologous virus population to achieve the desired effect. As a result, potent antiviral drugs that are able to, at least temporarily, suppress virus replication are required. These drugs are used in a specific manner to provide the optimal dose of antigen that can evoke the desired immune responses.

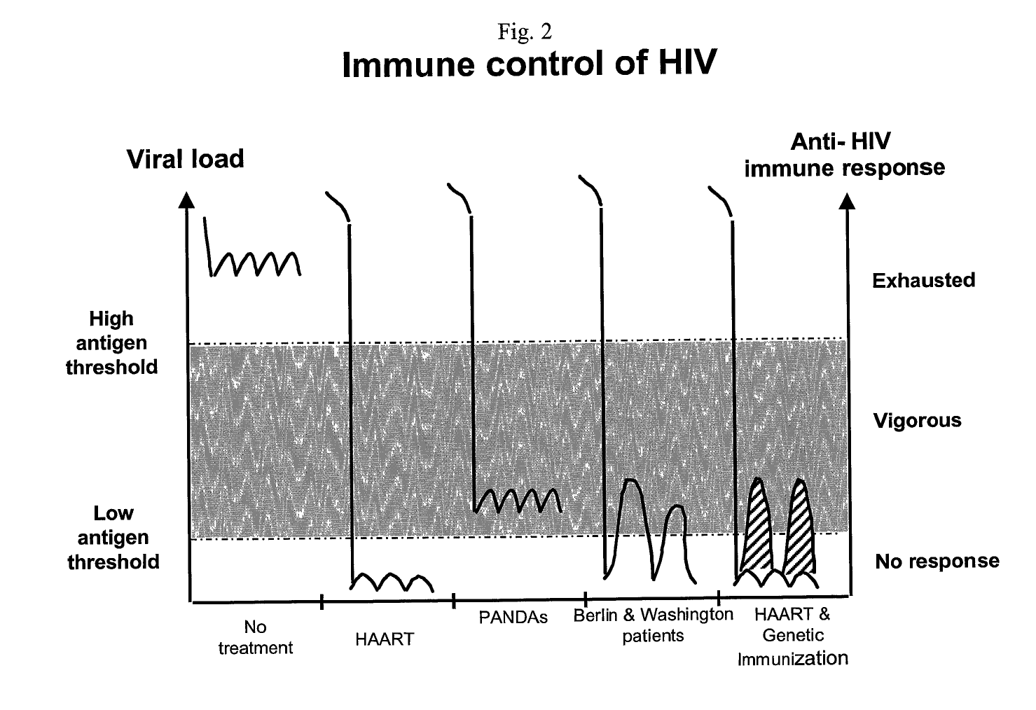

See FIG. 2 for a comparison of the viral load in the plasma of HIV-infected patients in various cohorts under various different treatment regimens. The first group is untreated patients. In that case, the initial HIV-1 infection may occur without accompanying symptoms, but most of the patients experience an acute HIV syndrome within 2 to 6 weeks of exposure to the virus. During this phase the virus replicates abundantly and is detectable in the blood.

Then, both the humoral and cellular arms of the immune system begin to respond to the infection. Antibodies to specific proteins associated with HIV-1 begin to appear in the serum between 2-12 weeks after primary infection. In addition, various types of immune system cells such as T-cells learn to recognize and destroy infected cells.

The combination of humoral and cellular immune responses together typically causes a decline of viral load in body fluids, or viremia, which ends the acute primary infection phase. In the absence of antiviral therapy, the immune system can partially control viremia, so that the numbers of viral particles in the body will drop and rise somewhat over many cycles, but generally remain high, that is, above the high antigen threshold. Over time, the immune system will eventually become exhausted. In the cases of patients treated with some continuous HAART regimens, the viral load begins at the untreated level, and then drops precipitously, below the low antigen threshold. The viral load does not stay in the desired range long enough to provoke an effective anti-HIV immune response. If treatment is stopped, the viral load of the patient will rebound, perhaps beyond the pretreatment level.

The portion of the figure labeled “PANDAS” (after a code name given to a cohort of patients) shows that, in the case of HIV, drugs that keep the HIV antigen level between the low and the high thresholds for longer periods of time can induce autovaccination, as discussed in more detail in U.S. Ser. No. 09/243,753, supra. Another strategy is to provide the optimal amounts of HIV antigen periodically to stimulate HIV-specific immune responses. As in the cases of the Berlin patient, the Washington patients and the monkey experiments described below in Examples 1 and 4, optimal doses of HIV antigen can be provided by STI.

The final portion of the figure demonstrates another method of inducing immune control of HIV, where a continuous HAART regimen is used to suppress viral replication, and an agent for activating quiescent cells, or more preferably, a vaccine for HIV, and most preferably a gene therapy vaccine encoding a replication incompetent form of HIV-1 as described in U.S. Ser. No. 08/803,484 filed Feb. 20, 1997 and in U.S. Ser. No. 09/153,198 filed Sep. 15, 1998, is administered to provide the dose of HIV antigen.

Structured Treatment Interruption (STI)

In one embodiment of the invention, described in more detail in Examples 1-3, STI involves short periods of drug treatments resulting in successful suppression of viral replication, preferably about 1-5 weeks, more preferably about 2-4 weeks, and even more preferably about 3 weeks. The treatment periods are followed by STOP periods, that is, periods in which no anti-HIV drugs are taken. The length of the STOP period is determined by the length of time until viral rebound. The goal of the STOP periods during autovaccination is to allow a controlled rebound of the autologous virus. This autologous virus can serve as an antigen that is present in the right amount to induce effective immune responses. In the case of HIV, the desired immune responses are T cell mediated immune responses.

In another preferred embodiment of the invention, described in more detail in Example 3, regular, symmetrical cycles of 2-4 weeks, preferably about 3 weeks on and 3 weeks off therapy, may be used. This embodiment has the advantage of simplicity, since viral load need not be monitored for the purpose of determining when to stop therapy. When this embodiment is used, it is often desirable that the first treatment period be extended. We have found that the initial period of drug therapy is preferably long enough to demonstrate that the therapy effectively inhibits virus replication to a level well below the high antigen threshold, and preferably below the low antigen threshold. The length of this initial treatment period will depend on the regimen used in the STI, and may be about 1-6 weeks. The number of cycles can vary, and the periods without therapy may be shifted from the limited, symmetrical form to the form where the period without therapy lasts until the viral load in the patient's tissues or fluids rebounds. Once the shift is made, the time to rebound will depend on the immune status of the patient. If the patient has already developed an effective immune response, he/she can stay without drugs for long time (months or end of his/her life). However, if the response is not present or is weak, the viral load will rebound and therapy has to be reinitiated.

Our animal data suggest 3-6 weeks of initial therapy can be followed with symmetrical cycles that include 3 week treatment periods and 3 week stop periods. More cycles might result in a better outcome. However, the number of cycles required depends on the status of the patient's immune system, especially upon the degree of activation of HIV specific cells, that is, whether the patient's T cell mediated immune responses are sufficient to control the virus. After complete cessation of drug therapy, the patients' viral load should be monitored periodically.

We have found that the degree of activation of HIV-specific cells during the successful therapy is time dependent and so, relatively short periods of therapy cannot be replaced with periods of long therapy combined with short interruptions. The optimal time to begin therapy interruption may be just shortly after the viral load reaches the 200-500 copies per ml (“undetectable”) level in the plasma.

The duration of autovaccination treatments depends in part on the overall immune status of the patient. If the patient is newly infected, for example if initial drug therapy is started before seroconversion is complete, the initial period of treatment is not more than about 1-5 weeks, and only about 2-10 cycles are needed. If the immune system is exhausted, a longer period, perhaps much longer (e.g. 6 months) of initial treatment might be suggested in order to restore those parts of the immune system that can be restored by HAART, that is, the portions that guard against opportunistic infections. In that case, a planned STOP period of preferably no more than one to three weeks might be adviseable to contain the burst of viremia that tends to accompany withdrawal of long-term HAART. Then an autovaccination sequence should be initiated, and the activation of HIV specific cells can develop via repeated drug treatment and STOP periods.

An alternative strategy is to initiate the treatment until the virus is undetectable (<400 copies/ml for at least 3 weeks) and follow up with short treatment periods combined with short STOP periods (for example 3 weeks treatment and 1 week stops). After rebound, it is important that the patients' viral load be decreased below the low antigen threshold during the treatment periods. To ensure that this occurs, the viral load is preferably reduced to less than about 200-500 copies/ml. If the viral load does not decrease, it may be desirable to change the antiviral drug therapy after the next stop period. It is important to note that STI is being used to ensure that the level of viral load is at some time within the appropriate range to induce effective viral inhibition, so the purpose here is to deliberately induce fluctuation in the viral load across the likely lower-to-middle end of the range effective to produce a useful immune response, while avoiding the overproduction of viral particles that can result in the infections of large numbers of immune system cells.

The presently recognized state of the art for HIV treatment is continuous drug treatment using HAART regimens that suppress HIV to undetectable levels in the patient's plasma. In order to maintain viral suppression most HAART regimens require that the patient comply exactly with the drug regimen. If the patient interrupts the therapy schedule, or if a drug resistant strain develops, the HIV will rebound. In addition, these drug regimens are powerful and may have toxic side effects when taken for long periods of time. Our monkey experiments described in Example 3, below, provide evidence that STI using HAART is a better way to treat HIV infected individuals than continuous HAART, because STI is less toxic, better tolerated, and as efficient for controlling both viral load and CD4 counts.

Assessing a Patient's Immune Competence

The pathogen-specific IFN-gamma assay described in Example 4 has been used by the inventors to distinguish between individual that have and have not been able to control viral rebound. This assay detects the percentage of activated, H IV-specific immune cells in a patient's blood or fluids. It is a very sensitive quantitative assay, because even low percentages represent large numbers of cells. The inventors have used this assay to distinguish between patients who have experienced rebound at a particular time and those who have not.

An average adult has about one million peripheral blood monocyte cells (PBMC) in one mililiter (ml) of blood. The total number of PBMC in a normal human body is about 10⁹ cells. 1% of these cells represents 100,000,000 cells and 0.1% represents 10,000,000. If we measure about1% HIV-specific T cells in a patient, the number of cells is about 100,000,000.

Alternatively, we can calculate the responding cells in one ml blood. There are about 1,000,000 PBMC per ml of blood. An individual with 1% HIV-specific T cells has about 10,000 HIV-specific cells, that is, cells able and ready to kill HIV-infected cells, in one ml of blood.

For the currently most common strains of HIV, one infected cell in the blood can produce about 200 HIV particles. If the viral load is one million copies/ml, about 5000 cells/ml are infected. If the viral load is 100,000 then 500 cells/ml produce HIV.

Therefore, if the viral load is one million in a patient who has 1% HIV-specific T cells, 10,000 guardian cells are trying to find and kill 5000 infected cells. If the patient has 100,000 or fewer copies/ml virus, 10,000 cells are available to destroy 500 or fewer infected cells. In both situations, the immune system is able to cope with the infection for several years, but cannot eliminate HIV because there is a continuous source of new infections.

This picture can be changed using the present inventions. For example, with 0.2% HIV-specific cells, a patient would have only about 2,000 cells per ml. If the initial viral load were one million then about 2,000 cells would be available to destroy 5000 infected cells (effector:target ratio 2:5) and some of those infected cells are likely to release nearly a full complement of viral particles (200 particles per cell). Given the rate of reinfection, it is clear that the immune system could eventually be overwhelmed.

After 3 weeks of successful drug treatment, the number of newly infected cells has decreased, and HIV-specific immune responses have increased. Consequently, an individual might now have 1% HIV-specific T cells, so that now 10,000 cells available to find and destroy the infected cells, and the immune system may be able to reach a new setpoint.

At this point, the initial viral load, meaning the viral load when the latest treatment period was begun, is significant. If the initial viral load was 1,000,000 copies/ml, the immune system must destroy about 5,000 cells (effector:target ratio of 2:1), but if the initial viral load was 100,000 copies/ml the immune system must destroy only 500 infected cells/ml , (effector: target ratio of 20:1). In addition, there are significantly fewer, or possibly no, newly infected cells. Where STI is repeated through several cycles, the net result may be a several-fold increase in HIV-specific immune responses and decrease in the number of infected cells. Further, where appropriate memory cells are stimulated, those cells may be available for use against future bursts of viremia.

Treatment Endpoint

In a preferred embodiment of the present invention, several cycles (e.g. 4) of STI are made before cessation of therapy. During STI, the patient's immune responses are developing and no “harm” is expected to occur. We have found that the patient's virus-specific immune response (VIR) as measured by a pathogen-specific IFN-gamma assay as described in Example 4 will suggest whether cessation of therapy is advisable. A higher percentage of HIV-specific immune system cells coupled with a very low viral load indicates that the immune system will be able to control viral replication for a longer period of time after therapy interruption.

If the patient's VIR is >2% at the time the patient's viral load is less then 400 copies/ml, the patient will able to control the virus after treatment interruption for a longer period of time. Additional STI cycles might increase the VIR several-fold. At that point autovaccination might be stopped. In this case the speed of rebound is zero or very slow. Patients are expected to behave as long term non-progressors. The viral load should be controlled by the immune system, and remain a low level (>5000 copies/ml) or undetectable by the standard blood test (<400 copies/ml), possibly after a contained rebound. Eventually, where the patient has very low viremia, that is, well below the limit of detectability of the standard blood tests, or less than 200 copies per ml, the patient's VIR may drop or become undetectable due to the absence of a sufficient amount of antigen.

The patient's viremia can still rebound if stress or other diseases disturb the immune system. Therefore, a patient's viral load, but not VIR, must be monitored periodically after the cessation of therapy, perhaps about every 6 months. Autovaccination must be restarted any time after the viral load significantly increases.

Patients must restart autovaccination using STI if the viral load increases and the immune system is no longer able to control viral replication. If the virus rebounds or the patient begins to suffer from other diseases, the antigen-specific immune responses are either decreased or have not been completely restored.

It is also possible that a given immune system is compromised at a late stage in disease development, and therefore an effective VIR response cannot develop. Even at this stage, an autovacciniation strategy might be beneficial to control virus replication and the development of escape mutants. Changing the drug regimen in the treatment cycles is suggested. For example: the treatment cycle AZT/3TC/lndinavir can be followed by a STOP period (e.g. 3 weeks); then treatment with ddl/HU/Nevirapin can be followed by a STOP period; then AZT/3TC/Indinavir can be reinitiated if no alternative combination available followed by a STOP period; then ddl/HU/Nevirapin might be used again, and so on.

This strategy provides maximal suppression of the virus during the treatment periods of STI, while the STOP periods provide a respite from the toxic effects of the drugs and may also mitigate the development of resistance.

Adjuvants for Autovaccination

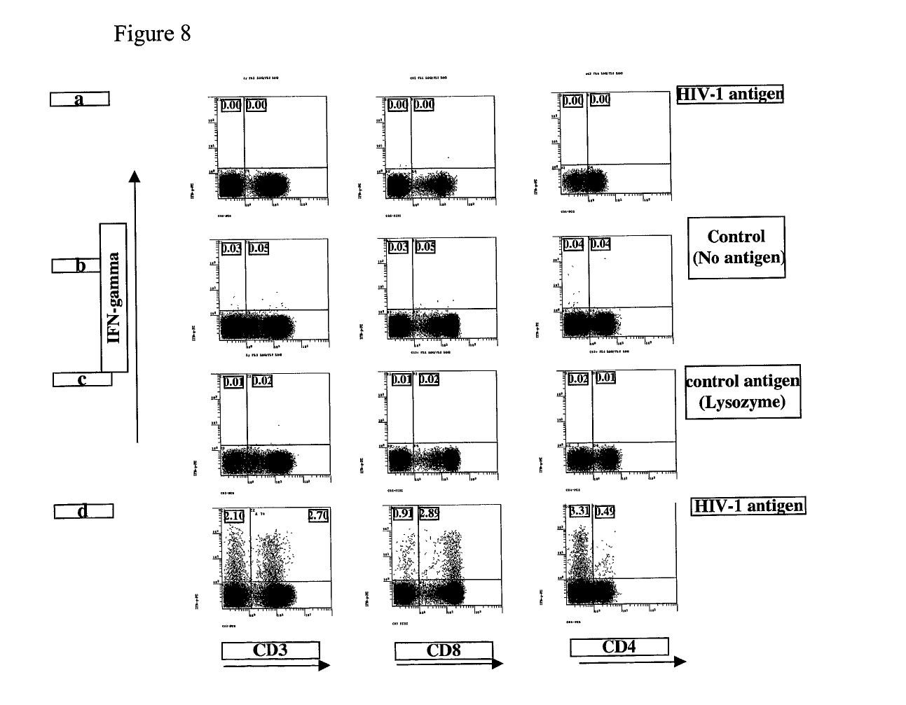

As we have discussed above, HIV-specific immune responses can control virus replication after interruption of therapy. Responses by HIV-specific IFN-gamma producing CD4 + and CD8+ T cells and non-T cells (such as natural killer, or NK, cells) can influence, or mediate, control of HIV by the immune system (non-T cell responses are also present in patient controlling virus as demonstrated in FIG. 8 in the CD3 negative population). These responses are generally called T helper immune responses of the Th1 subclass, or HIV-specific Th1 responses.

T helper immune responses might be divided into Th1 and Th2 type responses. Th1 responses elicit a strong cytotoxic T lymphocyte (CTL) mediated immune reactivity. Th2 responses elicit antibody production. The Th1/Th2 network is regulated by the interaction of a variety of different chemical messengers, or cytokines. The reciprocal stimulation/inhibition effects of these cytokines direct the type of immune responses.

The picture can be simplified as follows. Antigen presenting cells (mainly macrophages and dendritic cells) produce a cytokine, interleukin (IL) type 12. IL-12 stimulates Th1 cells, including both the T cells and natural killer (NK) cells. Another cytokine, IL-18, also functions as a strong activator of Th1 cells, particularly NK cells. Th1 cells mainly produce the cytokines IL-2 and interferon-gamma (IFNγ). These two cytokines in turn both stimulate macrophages to produce more IL-12 (positive feedback), and suppress the activity of Th2 cells. Th2 cells produce the cytokines IL-4 and IL-10. Both IL-4 and IL-10 inhibit macrophages and Th1 cells. In summary, Th1 and Th2 cells induce antagonistic immune responses. Therefore one would expect a Th1 response to be inhibited by the presence of Th2 cytokines, and vice versa.

HIV-1 is an intracellular pathogen. Intracellular pathogens are controlled by a Th1 type response. Consistent with this, Th1 immune responses were generated in the Berlin and Washington patients as well as in primate experiments to control HIV.

Therefore a further stimulation of a Th1 response, or inhibition of a Th2 response, or both, may contribute to the ability of the immune system to achieve control of HIV.

In general, all drugs or combinations of drugs that can stimulate Th1 responses and/or inhibite Th2 responses could be used as adjuvants. They might include, but are not limited to:

Stimulators of Th1 responses as adjuvants:

IL-12. IL-12 can stimulate IL-12 (autocrine loop) and IFNγ production. It can also increase II-15 and II-18. It stimulates Th1 T lymphocytes to secrete IFNγ and IL-2. It induces MHC class I and II presentation molecules in dendritic cells, and it stimulates the production of Th1 dependent antibodies (IgG2-alpha).

IL-2. Promotes T cell proliferation and inhibits production of Th2 cytokines.

Retinoids. They synergize with II-12 to stimulate IL-12 and IFNγ.

IL-18. II-18 synergizes with II-12 to induce IFN-gamma and to decrease IL-4.

IFNγ. It exerts a positive feed back on macrophages and inhibits Th2 responses.

Interferon α. It induces the IL-12 receptor, thereby favors a Th1 response. It stimulates CTL mediated responses. It decreases autoimmune (Th2 mediated) responses. It is produced by antigen presenting cells and acts on the same cells by inducing co-stimulatory molecules (i.e. B7).

Ribavirin. It stimulates IFNγ, IL-2 and TNF-α in vitro.

Fludarabin. It stimulates IFNγ, II-2 and inhibits IL-4 and II-10.

Inhibitors of Th2 responses as adjuvants:

Antibodies anti IL-10.

Antibodies anti IL-4.

Antibodies anti IL-5. IL 5 is another Th2 cytokine.

SB 203580 (Merck). It inhibits both IL-10 and IL-4.

Pooled human immunoglobulins. They inhibit IL-4 without major effects on IFN-gamma.

Suplatast tosilate. It prevents asthma by reducing IL-4 and II-10.

Suramin. It prevents binding of II-4 to its own receptor.

Teophillin. It reduces the amount of IL-4.

Corticosteroids. They inhibit IL-4 and II-5.

Some of these compounds might act as both stimulators of Th1 responses and inhibitors of Th2 responses.

Oligodeoxynucleotides (ODN) containing unmethylated CpG dinucleotides within specific sequence contexts (CpG motifs) are detected, like bacterial or viral DNA, as a danger signal by the vertebrate immune system. These and other sequences containing the CpG motif are known as both Th1 activators and Th2 inhibitors, and are therefore suitable as immunomodulatory adjuvants during autovaccination. [Hartmann G; Weeratna R D; Ballas Z K; Payette P; Blackwell S; Suparto I; Rasmussen W L; Waldschmidt M; Sajuthi D; Purcell R H; Davis H L; Delineation of a CpG Phosphorothioate Oligodeoxynucleotide for Activating Primate Immune Responses In Vitro and In Vivo. J Immunol Feb. 1, 2000;164(3):1617-1624 and Chiaramonte M G; Hesse M; Cheever A W; Wynn T A. CpG oligonucleotides can prophylactically immunize against Th2-mediated schistosome egg-induced pathology by an IL-12-independent mechanism J Immunol Jan. 15, 2000;164(2):973-85]

Adjuvants might be delivered by administration of the drug itself, a physiologically acceptable precursor, or DNA encoding the desired cytokine or its precursor. For example, injection of DNA encoding IFN-gamma can block the IL-10 response [Manickan E; Daheshia M; Kuklin N; Chun S; Rouse B T, Modulation of virus-induced delayed-type hypersensitivity by plasmid DNA encoding the cytokine interleukin-10.Immunology June 1998;94(2):129-34].

Another alternative way to induce Th1 responses is to inhibit gamma delta regulatory T cells [Seo N; Tokura Y; Takigawa M; Egawa K. Depletion of IL-10- and TGF-beta-producing regulatory gamma delta T cells by administering a daunomycin-conjugated specific monoclonal antibody in early tumor lesions augments the activity of CTLs and NK cells. J Immunol July 1, 1999;163(1):242-9]. When gamma delta T cells accumulate, their activities can attenuate the activity of CTLs and NK cells. Reducing the number of these cells may be useful whenever an effective Th1 response is desired. For example a daunomycin-conjugated anti-gamma delta TCR mAb UC7-13D5 (Dau-UC7) was shown to efficiently deplete gamma delta T cells and augment antigen-specific CTL as well as NK cell activities. These effects were mediated by the production of the two inhibiting cytokines IL-10 and TGF-beta by these cells.

Th1 stimulating or Th2 inhibiting adjuvants alone or in combination with antiretroviral therapy can be used to induce immune control of HIV. Similarly, Th1 stimulating or Th2 inhibiting compounds alone or in combination with antiviral and antitumor therapies can provide control of virus replication and tumor growth.

In a preferred embodiment, Th1 stimulating or Th2 inhibiting adjuvants are used in combination with STI. Example 8 shows that after successful antiretroviral therapy begins (1-6 weeks) Th1 immune responses increase in HIV-infected individuals. Treatment cycles including Th1 stimulating or Th2 inhibiting compounds are expected to further increase HIV-specific immune responses. This can happen by further stimulation of the primed cells (by, e.g. IL-2), by general activation of Th1 responses (by, e.g. IL-12), or by inhibition of Th2 responses and thereby indirectly increasing Th1 responses (by, e.g. anti-IL-4).

It is preferable to use immune stimulating drugs only after the viral load is reduced to undetectable levels (200-500 copies per ml) in order to limit the effects of the cytokines that are less desirable at the time. For example, some of these compounds might promote T cell proliferation or macrophage activation, thereby rendering these classes of cells temporarily more susceptible to HIV infection. It also may be particularly advantageous to administer immune stimulating drugs when viral load is low to compensate for the absence of antigen.

An immune stimulatory effect has been described in the case of the Berlin patient. This patient had a Hepatitis A infection. This is a massive infection that had to be eliminated by CTL responses induced against A. Therefore, Hepatitis A would have induced Th1 responses, such as the production of Th1 stimulating cytokines. In addition to inducing Hepatitis A-specific T cells to kill cells infected by the Hepatitis A virus, these cytokines may have unspecifically induced HIV-specific cells to kill HIV-infected cells. Such activity, called the bystander effect, is a recognized occurrence in immunology.

Since patients cannot be treated by Hepatitis A infection, we suggest that the same effect might be achieved by using Th1 stimulating or Th2 inhibiting compounds. These drugs should be given at the end of the treatment cycles of the STI, when viral load is low and immune response is high.

Data Supporting the Use of IL-10 Antagonists

Interferon-gamma (IFN-gamma) is considered useable as an indicator for both the Th1 response and CTL activity. Interleukin-10 (IL-10), on the other hand, has been shown to inhibit Th1-mediated immune responses. IL-10 is a significant IFN-gamma antagonist that mediates down-regulation of Th1-type cell-mediated immune responses. In other words, IL-10 appears to interrupt the beneficial activities of IFN-gamma. Recently, a clinical report indicated that high IL-10 levels in patients are associated with disease progression. Moreover, some viruses, such as Herpes viruses (Herpes simplex, CMV, HHV8, EBV and others), stimulate IL-10 production. Several reports have already demonstrated the role of these viruses as cofactor in HIV infection, however this mechanism for its action has not yet been proposed. An alternative hypothesis is that HIV-1 nef protein induces the production of IL-10. In any case, the correlation of viral replication and higher IL-10 production has not been described in HIV until now, but it has been reported that patients with progressive HIV infection have higher IL10 levels in the serum than non-progressors [Stylianou E; Aukrust P; Kvale D; Muller F; Froland S S Clin Exp Immunol April 1999;116(1):115-20]. This discovery further suggests that IL-10 levels should be also monitored in patients.

We have found that information from assays of the viral load, and intracellular production of both IFN-gamma and IL-10 is useful to monitor the patient's immune system. Together, these diagnostic assays can predict the efficacy of the Th1 immune responses to control HIV, which might be used to predict the new setpoint of viral load after interruption of therapy.

### SUMMARY OF THE INVENTION

The present invention relates to methods for treating disease caused by pathogens in the body. More specifically, it relates to methods of autovaccination, which is a technique for inducing antigen-specific immune cell responses. The inventors have found that highly active antiretroviral drug therapies (HAART) can be advantageously used in a method of treatment that includes structured treatment interruptions (STI). An advantage of the present invention is that it is as effective for reducing viral load in an infected patient as continuous HAART, while mitigating the amount of drugs used, and toxicity effects. Another advantage of the present invention is that it increases the ability of the infected patient's immune system to control a pathogen after treatment has stopped.

The present invention also relates to new methods of evaluating the status of a patient's immune system when a pathogen is present. The inventors have discovered that the percentage of pathogen-specific cells present in the body can be assessed by measuring the production of a cytokine, IFN-gamma, in a pathogen-specific test. This test, in conjunction with information about the patient's viral load, can be used to indicate whether a patient is able to stop therapy, at least temporarily. An advantage of the present invention is that it allows the practitioner to obtain more accurate information about the patient's immune system status. Another aspect of this invention is the discovery that the percentage of pathogen-specific cells present in the body is not constant, and fluctuation in the number of such cells can be used to identify optimal times for various treatments. Yet another aspect of the present invention is that the number of pathogen-specific cells in conjunction with information about the patient's recent history with respect to viral load can be used to estimate the relative numbers of effector and infected cells.

The present inventors have also discovered that intracellular levels of the cytokines IFN-gamma and IL-10, produced by PBMCs from HIV-1-infected individuals, correlate with the patient's ability to suppress viral replication. Hence, one advantage of the invention is that changes in the amount of viral replication can be more easily predicted.

The present inventors have discovered that when the percentage of PBMC producing INF-gamma is higher than the percentage of PBMC producing IL-10, in conjunction with a low viral load, then a patient's immune system may be able to control viral replication without antiviral therapy. An advantage of this invention is that it provides a means to both increase the amount of INF-gamma and lower the amount of IL-10 in the immune system at a given time, and thereby shift the percentage of cells engaged in IFN-gamma production and IL-10 production to advantageous levels.

Another aspect of this invention is a diagnostic test, useful to predict whether the patient's immune system can suppress viral replication when drug therapy is stopped. Specifically, the inventors have discovered that when the viral load is low, preferably less than about 200-500 copies per ml, and IFN-gamma production is higher than IL-10 production, then the patient is in the best position to stop taking antiviral therapy, at least on an intermittent basis.

Another aspect of this invention entails using compositions able to inhibit IL-10 production such as an anti-IL-10 antibody, to block the activity of IL-10 in vivo. This can be used to suppress active levels of IL-10 below IFN-gamma and maintain a good immune response, resulting in a benefit to the patient.

A specific embodiment of this invention is a diagnostic method to determine if a patient should go off antiviral therapy, comprising the steps of measuring viral load and measuring IFN-gamma and IL-10 production, such that if the viral load is less than or equal to 500 copies/ml and if IFN-gamma is higher than IL-10, then the patient can cease antiviral therapy for the period of one week. Yet another advantage of the present invention is that, when immuno-modulatary therapies as described herein are used, the immune system may acquire the ability to control HIV for long periods of time more rapidly, and the total number of STI cycles might be reduced.

Drugs Suitable for Autovaccination and in STI

Current antiretroviral drug regimens typically rely on one or more reverse transcriptase inhibitors, protease inhibitors, and a variety of other drugs including immune system treatments and a variety of unique agents, and may include 2-4 (or more) compounds, administered together. Any combination of the drugs described below might be used in an STI-style treatment for HIV infection. Hydroxyurea-containing combinations are preferred.

Hydroxyurea is one of many inhibitors of ribonucleotide reductase, an enzyme known for catalyzing the reduction of ribonucleoside diphosphates to their deoxyribonucleoside counterparts for DNA synthesis. Hydroxyurea inhibits viral replication, and also acts to down-modulate the immune system. Another material that inhibits viral replication and down-modulates the immune system is cyclosporine, a cyclophilin inhibitor. Other ribonucleotide reductase inhibitors include guanazole, 3,4-dihydroxybenzo-hydroxamic acid, N,3,4,5-tetrahydroxybenzimidamide HCl, 3,4-dihydroxybenzamidoxime HCl, 5-hydroxy-2-formylpyridine thiosemicarbazones, and n-(N)-heterocyclic carboxaldehyde thiosemicarbazones, 4-methyl-5-amino-1-formylisoquinoline thiosemicarbazone, N-hydroxy-N′-amino-guanidine (HAG) derivatives, 5-methyl-4-aminoisoquinoline thiosemicarbazone, diaziquone, doxorubicin, 2,3-dihydroxylbenzoyl-dipeptides and 3,4-dihydroxylbenzoyl-dipeptides, iron-complexed 2-acetylpyridine 5-[(2-chloroanilino)-thiocarbonyl]-thiocarbonohydrazone (348U87), iron-complexed 2-acetylpyridine-5-[(dimethylamino)thiocarbonyl]-thiocarbonohydrazone (A1110U), 2′-deoxy-2′-methylenecytidine 5′-diphosphate (MdCDP) and 2′-deoxy-2′, 2′-difluorocytidine 5′-diphospahte (dFdCDP), 2-chloro-9-(2-deoxy-2-fluoro-β-D-arabinofuranosyl)-adenosine (Cl—F-ara-A), diethyidithiocarbamate (DDC), 2,2′-bipyridyl-6-carbothioamide, phosphonylmethyl ethers of acyclic nucleoside analogs, [eg. diphosphates of N-(S)-(3-hydroxy-2-phosphonylmethoxypropyl and N-2-phosphonylmethoxyethyl) derivatives of purine and pyrimidine bases], nitrosourea compounds, acylclonucleoside hydroxamic acids (e.g., N-hydroxy-n-(2-hydroxyethoxy)-1(2H)-pyrimidineacetamides 1-3, and 2-acetylpyridine 4-(2-morpholinoethyl)thio-semicarbazone (A723U)).

In human chemotherapy, hydroxyurea is currently administered using two basic schedules: (a) a continuous daily oral dose of 20-40 mg per kg per day, or (b) an intermittent dose of 80 mg per kg per every third day. Either schedule could be used in the treatment of viral infections. Lower dosages of hydroxyurea may also be effective in treating HIV infections. The presently preferred dosage range for use of hydroxyurea in treating HIV infections is 800-1500 mg per day, which can be divided over a 24 hour period, for example as 300-500 mg three times a day (TID), 500 mg twice a day (BID), or 1,000 mg once a day (QD), assuming an adult weighing about 70 kg. When the patient's weight is over 60 kg, 400 mg TID is preferred, for those under 60 kg, 300 mg TID is preferred.

Reverse transcriptase inhibitors figure prominently in current HIV treatments. Examples include nucleoside analogs, such as the 2′,3′-dideoxyinosine (ddl)(available as Videx® from Bristol Myers-Squibb). Nucleoside analogs are a class of compoounds known to inhibit HIV, and ddl is one of a handful of agents that have received formal approval in the United States for clinical use in the treatment of AIDS. Like zidovudine (3′-azido-2′,3′-dideoxythymidine or azidothymidine [AZT] available from Glaxo Wellcome), zalcitabine (2′,3′-dideoxycytidine [ddC] available as Hivid® from Hoffman-La Roche), lamivudine 2′-deoxy-3′-thiacytidine [3TC](Epivir® available from Glaxo Wellcome), Iodenosine (F-ddA available from US Biosciences and stavudine (2′,3′-didehydro-2′,3′-dideoxythimidine [D4T] available as Zerit® from Bristol Myers-Squibb), ddl belongs to the class of compounds known as 2′,3′-dideoxynucleoside analogs, which, with some exceptions such as 2′,3′-dideoxyuridine [DDU], are known to inhibit HIV replication, but have not been reported to clear any individual of the virus. Other nucleoside reverse transcriptase inhibitors include adefovir (Preveon® an adenine nucleotide analog from Gilead Sciences), abacavir (1592U89 available from Glaxo Wellcome), lubocavir (a guanosine analog available from Bristol Meyers-Squibb), and PMPA, available from Gilead Pharmaceuticals. New nucleosides include FTC (Emtricitabine), DAPD, also known as DXG, F-ddA (Lodenosine, a fluorinated purine nucleoside RTI, and dOTC (BCH-10562). Non-nucleoside reverse transcription inhibitors include nevirapine (Viramune™ available from Boehringer Ingelheim Pharmaceuticals, Inc.), delaviridine (Rescriptor® available from Pharmacia & Upjohn) and efavirenz (available as Sustiva®, from DuPont Merck) Currently, antiviral therapy requires doses of ddl at 200 mg per day BID for an adult human, or in the alternative 400 mg once a day (QD). Similar dosages may be used in the present invention. However, use of combinations of drugs may increase the effectiveness of these nucleoside phosphate analogs so that they can be used at lower dosages or less frequently. In combination with hydroxyurea, the presently preferred range for ddl is 100-300 mg twice a day (BID) or 400 mg once a day (QD), assuming an adult weighing 70 kg. When d4T is used with either hydroxyurea or a combination of hydroxyurea and ddl, the preferred range is 40 mg BID.

Of the potential protease inhibitors for use against HIV, compounds such as hydroxyethylamine derivatives, hydroxyethylene derivatives, (hydroxyethyl)urea derivatives, norstantine derivatives, symmetric dihydroxyethylene derivatives, and other dihydroxyethylene derivatives have been suggested, along with protease inhibitors containing the dihydroxyethylene transition state isostere and its derivatives having various novel and high-affinity ligands at the P2 position, including 3-tetrahydrofuran and pyran urethanes, cyclic sulfolanes and tetrahydrofuranylglucines, as well as the P3 position, including pyrazine amides. In addition, constrained “reduced amide”-type inhibitors have been constructed in which three amino acid residues of the polypeptide chain were locked into a g-turn conformation and designated g-turn mimetics. Other alternatives include penicillin-derived compounds and non-peptide cyclic ureas. Buitable protease inhibitors include Indinavir sulfate, (available as CrixivanTM capsules from Merck & Co., Inc, West Point, Pa.), saquinavir (Invirase® and Fortovase® available from Hoffman-LaRoche), ritonavir (Norvir® available from Abott Laboratories) ABT-378 (available from Abott Laboratories), Nelfinavir (Viracept®), and GW141 (available from Glaxo WellcomeNertex) Tipranavir available from Pharmacia & Upjohn, PD 178390 available from Parke-Davis, BMS-23632 available from Bristol-Myers Squibb, DMP-450 available from Triangle, and J E 2147 available from Agouron. New protease inhibitors include ABT-378 (Abbott laboratories), L-756423, DMP-450 and AG1776.

In addition to reverse transcriptase inhibitors and protease inhibitors, the present invention may utilize integrase inhibitors such as AR177 (Zintenvir® available from Aronex); fusion inhibitors such as pentafuside, (T-20) and cytokine inhibitors (available from Chiron), chemokine inhibitors, and antisense oligonucleotides such as GPI-2A available from Novopharm Biotech, ISIS-13312 available from Isis, and GEM-132 and GEM-92 available from Hybridon. Other compounds which might be used include mycophenolic acid (MPA, available from Celicept) and PRO 52 (CD4-Ig2), a fusion protein comprising a human immune globulin in which parts of the heavy and light chains have been replaced with domains from the human CD4+ cell.

Suitable human dosages for these compounds can vary widely. However, such dosages can readily be determined by those of skill in the art. For example, dosages to adult humans of from about 0.1 mg to about 1 g or even 10 g are contemplated.

### SUMMARY OF THE INVENTIONS

The present invention relates to methods for treating disease caused by pathogens in the body. More specifically, it relates to methods of autovaccination, which is a technique for inducing antigen-specific immune cell responses. The inventors have found that highly active antiretroviral drug therapies (HAART) can be advantageously used in a method of treatment that includes structured treatment interruptions (STI). An advantage of the present invention is that it is as effective for reducing viral load in an infected patient as continuous HAART, while mitigating the amount of drugs used, and toxicity effects. Another advantage of the present invention is that it increases the ability of the infected patient's immune system to control a pathogen after treatment has stopped.

The present invention also relates to new methods of evaluating the status of a patient's immune system when a pathogen is present. The inventors have discovered that the percentage of pathogen-specific cells present in the body can be assessed by measuring the production of a cytokine, IFN-gamma, in a pathogen-specific test. This test, in conjunction with information about the patient's viral load, can be used to indicate whether a patient is able to stop therapy, at least temporarily. An advantage of the present invention is that it allows the practitioner to obtain more accurate information about the patient's immune system status. Another aspect of this invention is the discovery that the percentage of pathogen-specific cells present in the body is not constant, and fluctuation in the number of such cells can be used to identify optimal times for various treatments. Yet another aspect of the present invention is that the number of pathogen-specific cells in conjunction with information about the patient's recent history with respect to viral load can be used to estimate the relative numbers of effector and infected cells.

The present inventors have also discovered that intracellular levels of the cytokines IFN-gamma and IL-10, produced by PBMCs from HIV-1-infected individuals, correlate with the patient's ability to suppress viral replication. Hence, one advantage of the invention is that changes in the amount of viral replication can be more easily predicted.

The present inventors have discovered that when the percentage of PBMC producing INF-gamma is higher than the percentage of PBMC producing IL-10, in conjunction with a low viral load, then a patient's immune system may be able to control viral replication without antiviral therapy. An advantage of this invention is that it provides a means to both increase the amount of INF-gamma and lower the amount of IL-10 in the immune system at a given time, and thereby shift the percentage of cells engaged in IFN-gamma production and IL-10 production to advantageous levels.

Another aspect of this invention is a diagnostic test, useful to predict whether the patient's immune system can suppress viral replication when drug therapy is stopped. Specifically, the inventors have discovered that when the viral load is low, preferably less than about 200-500 copies per ml, and IFN-gamma production is higher than IL-10 production, then the patient is in the best position to stop taking antiviral therapy, at least on an intermittent basis.

Another aspect of this invention entails using compositions able to inhibit IL-10 production such as an anti-IL-10 antibody, to block the activity of IL-10 in vivo. This can be used to suppress active levels of IL-10 below IFN-gamma and maintain a good immune response, resulting in a benefit to the patient.

A specific embodiment of this invention is a diagnostic method to determine if a patient should go off antiviral therapy, comprising the steps of measuring viral load and measuring IFN-gamma and IL-10 production, such that if the viral load is less than or equal to 500 copies/ml and if IFN-gamma is higher than IL-10, then the patient can cease antiviral therapy for the period of one week. Yet another advantage of the present invention is that, when immuno-modulatary therapies as described herein are used, the immune system may acquire the ability to control HIV for long periods of time more rapidly, and the total number of STI cycles might be reduced.

### EXAMPLES

The following examples illustrate the practice of various aspects of the present inventions. They do not limit the inventions, or the claims, which follow them.

### Example 1

### Feasibility: STI and a Specific Drug Regimen

We have recently described the case of a Berlin patient treated with hydroxyurea, didanosine, and a protease inhibitor before complete Western Blot (WB) seroconversion, who interrupted the treatment two times before permanent discontinuation. Two years after therapy discontinuation, the level of HIV RNA in the plasma of this patient was still below the limit of detection (<500 copies/ml). This case suggested that structured treatment interruptions (STI) of hydroxyurea based combinations might contribute to the control of HIV replication in the absence of drugs.

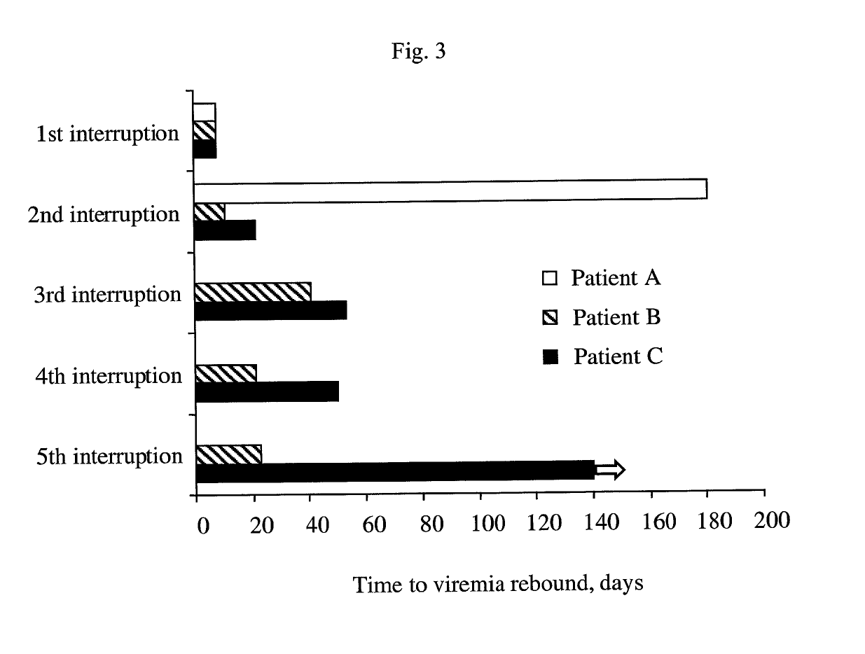

To test the feasibility of STI in a prospective study, three patients who had never had antiretroviral drug therapy were treated with hydroxyurea-containing HAART at varying time points after complete WB seroconversion. The patients were heterogeneous with respect to initial viral load and CD4 T cell count. Baseline values were 16, 130, 21,845, and 719,000 RNA copies/ml, and CD4 count was 508, 264, and 880, in patients A, B, and C, respectively. Patients were scheduled to be treated for 3 weeks, followed by 1 week interruption, in order to mimic the case of the Berlin patient. Subsequently, patients were treated with consecutive cycles of ca. 3 months of treatment followed by treatment interruption. Patient viremia was monitored weekly when therapy was withheld. Following the second to the fifth treatment interruption, therapy was scheduled to be restarted when viremia rebounded above the threshold of 5,000 copies/ml. The time required for HIV to rebound is described in FIG. 3. Patient A maintained a level of viremia around 2,000 copies/ml (approximately 1 log lower than baseline) for 6 months during the second interruption. Subsequently, viremia rebounded to 6,919 copies/ml and the patient restarted therapy. Time to rebound increased in patient B during the second and third interruption, however, not during the fourth and fifth interruption. In patient C, time to rebound increased during the second and third interruption, however, not during the fourth interruption, similar to patient B. In contrast, viremia remained below 5,000 copies/ml (2 to 3 logs lower than baseline values) as long as 5 months during the fifth interruption. CD4 count at the end of the follow up was 690, 435, and 630 respectively. Importantly, therapy rapidly reduced viremia below limit of detection after each restart.

These results suggest that hydroxyurea-based combinations can be interrupted and successfully restarted several times. HIV could be controlled after each therapy restart, suggesting that no drug resistance emerged after as many as five consecutive therapy discontinuations. CD4 count increased in two patients and decreased in the third one. A lower viremia set point could be induced and maintained for several months in two of three patients. A third patient failed to acquire a prolonged control of HIV in the absence of therapy. These initial findings extend our original observation, and suggest that under some circumstances antiretroviral therapy is able to induce a transient control of HIV after therapy discontinuation, even if treatment is begun after complete WB seroconversion. However, achieving control of HIV in the absence of therapy might require several STI cycles in some patients and might not be successful in others. Therefore, it is important to precisely ascertain the mechanisms of HIV control after treatment interruption, and to identify the correlates that predict the control of viremia.

### Example 2

### Searching for Correlates

In this experiment we studied monkeys that had been infected with a pathogenic Simian Immunodeficiency (SIVmac251) more than one year. Before treatment, the viral load of one monkey (624) was 20,854 copies/ml and the viral load of the other monkey (652) was 1,757,901 copies/ml. Animal 652 had symptomatic AIDS. Both animals were treated with HAART (HU+ddl+PMPA) for one month. After 3 weeks of therapy the viral load of both animals became undetectable (<5000 copies/ml). Following the one month of treatment, therapy was interrupted. Monkey 624 was able to control SIV replication for one month, then he rebounded to 41,341 copies/ml. Interestingly, after this rebound SIV was spontaneously controlled again by the immune system and the monkey became undetectable again. In contrast, SIV rebounded in monkey 562. Seven days after interruption of therapy his viral load was 21,040 copies/ml, and 2 weeks later over 3 million copies/ml. Antiretroviral therapy for this animal was restarted. SIV-specific immune responses were analyzed as described in Example 4, below, before therapy interruption and the results are summarized in Table I, below.

TABLE I
Immune responses of chronically-infected monkeys

| Oct. 20 | | 624 | 624 | 562 | 562 |
|---|---|---|---|---|---|
| VL | | <5000 | | <5000 | |
| IFN-gamma | | C1 or C2 | C2/C2 + C4 | C1 or C2 | C2/C2 + C4 |
| | | % total | % specific | % total | % specific |
| media | CD3− | 0.27 | | 0.12 | |
| | CD3+ | 0.29 | 0.35 | 0.22 | 0.28 |
| | CD3+ CD8− | 0.16 | 0.41 | 0.05 | 0.11 |
| | CD3+ CD8+ | 0.37 | 0.61 | 0.17 | 0.3 |
| SIV | CD3− | 1.11 | | 0.28 | |
| | CD3+ | 1.82 | 2.22 | 0.51 | 0.66 |
| | CD3+ CD8− | 1.03 | 2.58 | 0.22 | 0.49 |
| | CD3+ CD8+ | 1.45 | 2.41 | 0.39 | 0.7 |

Four different classes of cells that might have a role in controlling HIV were assayed for the percentage of activated, SIV-specific cells. The results are shown in Table I. CD3− cells are non-T-cells that may play an important role in eliminating pathogens, of these, a subset would be NK cells. CD3+ cells include all cells having SIV-specific T cell responses. CD3+CD8− cells include the SIV-specific T helper population, and more than 80% of these cells are CD4+ T cells. CD3+CD8+ cells are cytotoxic T cells (CTL).

The monkey that was able to control viral rebound (624) had larger numbers of activated cells in all categories than the monkey (562) that could not control viral rebound after therapy interruption. While the monkey able to control viral rebound (624) was generally healthier than the other monkey, the disparity in initial viral load may have been a significant factor in the outcome. Based on the initial viral load, monkey 562 would have had about 100 times, or 2 log more infected cells than monkey 624.

### Example 3

### Monkey Trial Comparing STI to Continuous HAART

The infection of rhesus macaques by Simian Immunodeficiency Virus (SIVmac251) was chosen as an animal model because of the similarities of SIV in macaques to HIV infection in humans. Mucosal inoculation of macaques with SIVmac251 reproducibly resulted in an infection characterized by peak plasma viremia within 2-3 weeks post infection, followed by a plateau which can persist for several months. Eventually, most animals progress toward an acquired immune deficiency syndrome, although, occasionally, a low percentage of infected animals manage to spontaneously control virus replication and exhibit very low levels of plasma viremia, similar to human long-term non progressors. Studies of antiretroviral therapy have been limited until recently when PMPA was shown to effectively inhibit SIV replication in this non-human primate model. Protease inhibitors do not work in an SIV infected monkey model. Therefore, we have used the combination of PMPA, ddl and HU as HAART, because our preliminary experiments demonstrated that this combination can rapidly and effectively decrease viral load in SIV-infected animals.

A total of 29 rhesus macaques were infected via mucosal (intra-rectal) inoculation with SIVmac251 (5.12×10³ TCID 50 in 3ml). The combination of PMPA (20 mg/Kg once daily subcutaneously), ddl (10 mg/Kg once daily intravenously), and HU (15 mg/Kg once daily intravenously) was selected because preliminary experiments had shown that this combination can effectively suppress SIV viral load for long periods of time, similar to HAART in HIV infected humans. A group of five SIV infected and untreated animals served as controls. A group of six SIV infected animals received continuous antiretroviral therapy initiated 44 days post infection. The other 3 groups were treated intermittently for a total of 24 weeks. The groups treated intermittently were on the same schedule, 3 weeks on followed by 3 weeks off. In sum, Group # 1 was untreated, Group #2 was treated with intermittent therapy, (HU+ddl+PMPA); Group #3 was treated with intermittent therapy that did not include hydroxyurea, (ddl+PMPA); Group #4 was treated with intermittent therapy for two drugs, ddl and PMPA, and continuous therapy for a third, hydroxyurea (ddl+PMPA, intermittent, HU continuous); Group #5 was treated continuous therapy (HU+ddl+PMPA, continuous treatment).

STI is Superior to HAART for Treatment of Viral Diseases

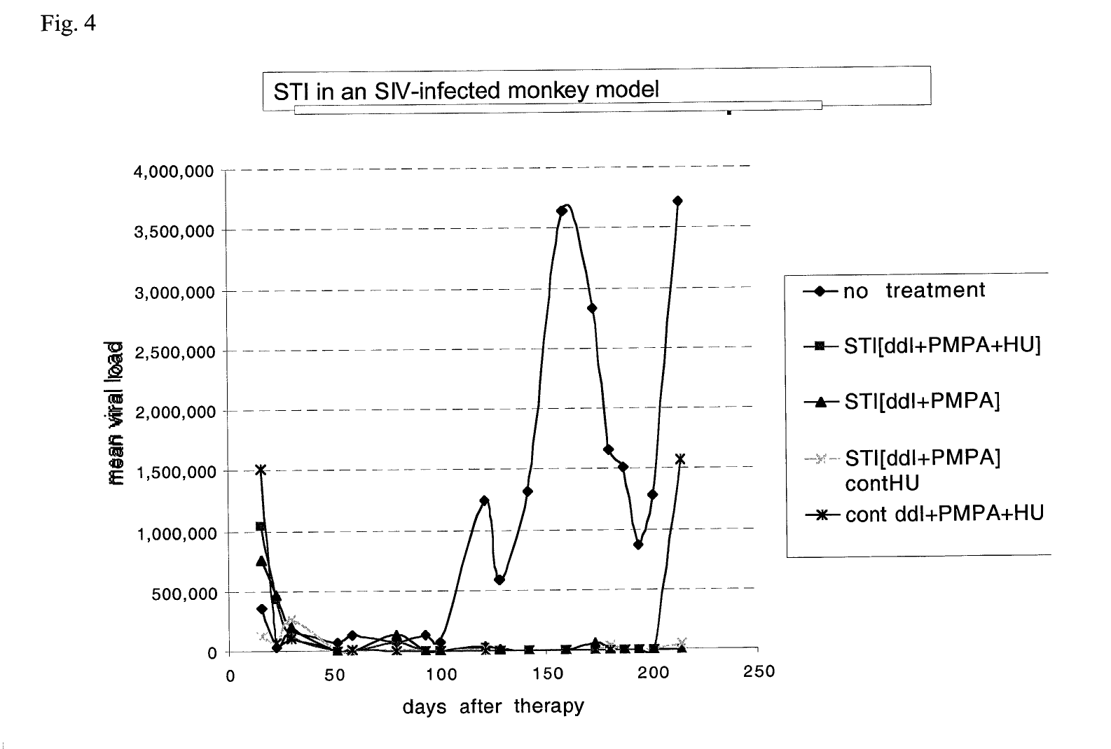

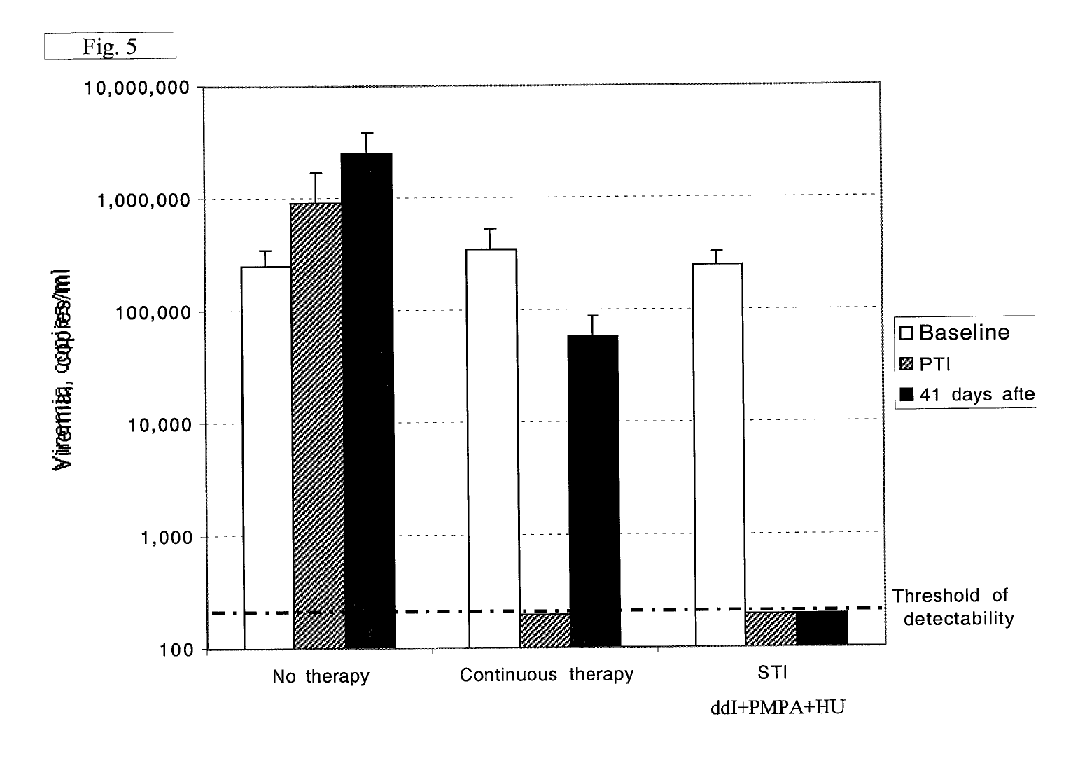

FIG. 4 shows the viral load for all monkeys from shortly before therapy was begun until about one month after therapy ended. FIG. 5 shows the viral load for the same monkeys at baseline, during therapy, and 41 days after cessation of therapy. Because their results are similar, all the monkeys treated with STI are shown in FIG. 5 as a single group. The virology of this experiment demonstrates that both treatment schedules, continuous HAART and STI, decreased the viral load efficiently after introduction of therapy. Compared to the untreated control, the viral load in all cases was either undetectable or at a very low level during the treatments. The differences among the three STI therapies with respect to the maintenance of viral load were also insignificant during the treatment.

This picture changed dramatically after permanent treatment interruption. The viral load of the animals rebounded in the group treated continously with HAART (cont ddl+PMPA+HU) and one animal died one month after therapy interruption. No animals died in the untreated control group. This was not surprising, because it is known that after interruption of HAART, viral load rebounds to the pretreatment values or higher, even if it starts from a very low undetectable level. In contrast, the monkeys treated with STI controlled SIV replication at least 2 months after permanent interruption of therapy. In the group of STI(ddl+PMPA+HU) the results were dramatic: 6 of the 6 animals controlled SIV. In each of the two other groups of STI(ddl+PMPA) and STI(ddl+PMPA+cont HU) one animal was a non-responder (never respond to therapy, a finding not uncommon in the treatment of these animals, irrespective of the kind of treatment administered), one animal's viral load rebounded, and 4 animals controlled SIV. These results demonstrate that (1) continous HAART cannot be interrupted because viral load rebounds rapidly and, more importantly, after therapy interruption, patients have a higher risk of dying than if they had remained untreated; (2) Intermittent therapy (STI) can control viral replication after therapy discontinuation; (3) Hydroxyurea is a useful but not essential component of HAART used for STI.

CD4 Counts

A patient's CD4 counts typically decrease if HIV infection is untreated. One concern with HU-containing therapies was that although these therapies decrease the viral load, significant increases in the patient's CD4+ T cell count are generally not observed. Here we studied CD4 counts in our monkey model.

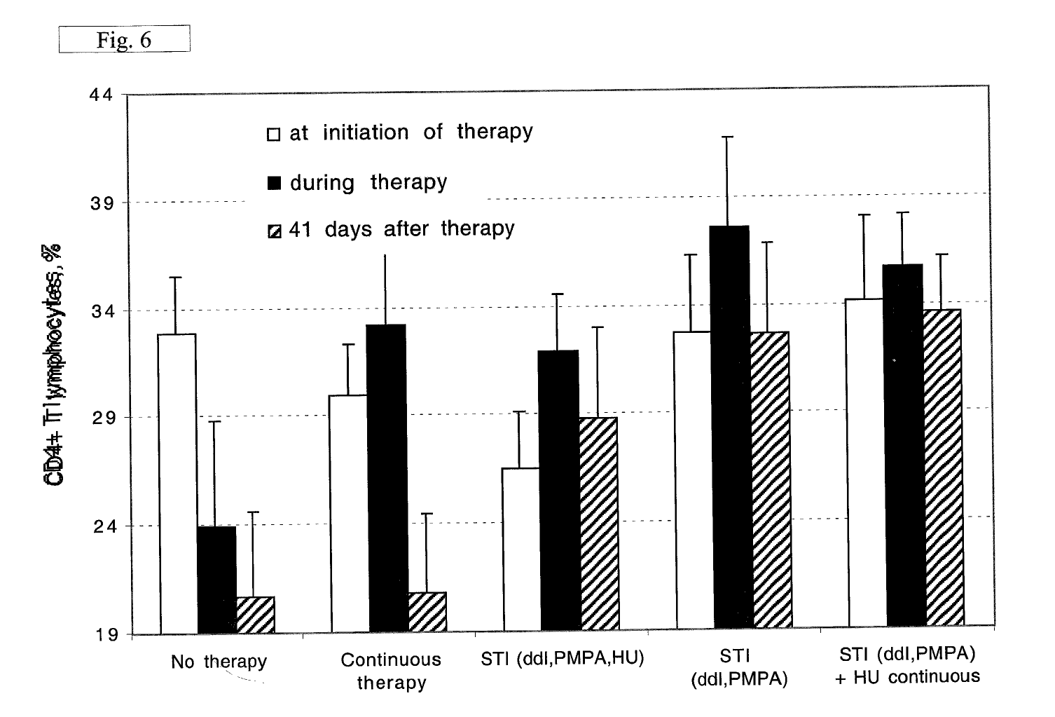

FIG. 6 shows the number of CD4+ lymphocytes for 29 monkeys at initiation of therapy, during therapy, and 41 days after cessation of therapy.

Our results confirm that the course of infection in the monkey model is similar to that of HIV in that CD4 cell counts consistently decrease over time during SIV infection in the absence of treatment. Both continuous HAART and STI can increase the CD4 count. At the end of therapy, no differences in CD4 cell counts were observed between the continuous HAART and STI groups. This is consistent with the viral load analysis and provides further evidence that that STI is as effective for treatment as continuous HAART.

After cessation of continuous HAART, the CD4 counts began to decrease rapidly. At 41 days after treatment interruption, the CD4 counts of animals treated with continuous HAART were no different from the CD4 counts of untreated animals. The CD4 count and the viral load data provide evidence that patients treated with continuous HAART lose the benefits gained during therapy. Since we had one death in the HAART group, it is also possible that continuous HAART treatment is worse than no treatment if the therapy has to be permanently interrupted, as might be the case when a drug has toxic side effects. It is notable that, 41 days after permanent discontinuation of STI, the CD4 counts had not decreased significantly. This data provides additional evidence that a virus can be controlled after permanent discontinuation of STI.

STI is Less Toxic than Continuous HAART

Bone marrow toxicity, known to be associated with the use of HU, was closely monitored. Two animals in the continuous HAART group experienced a slight decrease in the hemoglobin levels (from 11.9 g/dL to 9.2 g/dL in animal #19196, and from 13.1 g/dL to 11.1 g/dL in animal #19152). This mild toxicity was attributed to the use of HU, however, it did not warrant any modification of HU dosage. Bone marrow toxicity was more severe in the untreated controls. In three animals (#716, #19763, and # 19766) hemoglobin levels decreased from 13.1 g/dL to 10.5 g/dL, from 12.8 g/dL to 9.2 g/dL, and from 12.8 g/dL to 8.0 g/dL, respectively. This decrease probably reflected the SIV-mediated bone marrow toxicity. Unexpectedly, five months after treatment initiation, technicians noted a visible decline in the health of some animals treated with continuous HAART therapy. Blood tests revealed that five of six animals had increased glucose levels in the plasma (range 231 to 448 mg/ml) and one of them (animal #19197) also had increased transaminase levels (AST=448 IU/L, and ALT=382 IU/L). In contrast, the infected untreated animals had normal values, indicating that the toxicity was due to the use of the antiretroviral drugs.

Administration of all drugs was interrupted for the group. Despite the treatment interruption, the clinical conditions deteriorated. Two animals (#19197 and#19720) were reported as depressed in their cages, hunched, and anorexic. They also had weight loss of 0.5 to 1.0 Kg from the previous week, mainly due to dehydration. Both animals received fluid therapy and nutritional support. Two other animals (#19196 and #19512) had abdominal distension, visible wasting and increased output of clear urine. Two weeks after treatment interruption, glucose levels in five of six animals (#19720, #19197, #19152, #19196, and #19729) were severely elevated (758-1455 mg/ml) (Table II). Four of these animals had increased aspartate aminotransferase (AST) levels (245-10450 IU/L), and three also had increased alanine aminotransferase (ALT) levels (246-780 IU/L). Two of the animals showed increased amylase levels (746 and 2134 IU/L). Alkaline phosphatases were very high in one animal (3090 IU/L) and moderately elevated in another one (793 IU/L). Lipases were significantly elevated in one animal (623 IU/L), and slightly elevated in two other animals (Table II). The sixth animal (#710) had glucose levels slightly above normal (108 mg/ml), and amylases were also elevated (746 IU/L). Insulin treatment (recombinant-human DNA derived, at 2 lU/Kg) was promptly started in all animals, except in animal # 710. One week later the conditions of the two animals (#19720 and#19197) that were most ill stabilized. The animals became eager to eat and much more active. Glucose and transaminase levels significantly decreased. All five animals exhibited severe muscle wasting, weakness polyuria and polydypsia. Insulin treatment, nutritional and supportive care were continued. Note, that these are common toxicities in HAART treated patients.

TABLE II
Laboratory values of 6 rhesus macaques continuously treated
with PMPA, ddl and HU
2 weeks after treatment interruption

| Animal # | 19720 | 19197 | 19152 | 19196 | 19729 | 710 | (Normal) |
|---|---|---|---|---|---|---|---|
| GLU | 1455 | 977 | 950 | 758 | 908 | 108 | 33-95 |
| ALT | 780 | 473 | 129 | 246 | 188 | 41 | 18-204 |
| AST | 10450 | 245 | 124 | 596 | 373 | 50 | 23-175 |
| ALK P | 793 | 3090 | 304 | 231 | 239 | 526 | 65-641 |
| LDH | 1705 | 498 | 575 | 749 | 1326 | 747 | 578-4603 |
| TRIG | 44 | 60 | 218 | 164 | 79 | 83 | 23-194 |
| AMYL | 137 | 139 | 2134 | 495 | 357 | 746 | 178-551 |
| LIP | 222 | 363 | 623 | 167 | 181 | 130 | 30-190 |

In striking contrast to what happened in these animals, no toxic side effects were observed in any of the animals that received STI treatment.

### Example 4

### The Washington Patient

The rationale for STI is to schedule interruptions of treatment to allow a controlled virus rebound. STI might offer advantages over continuous treatment: substantial periods of treatment interruptions might lead to a reduced toxicity and improved quality of life. However, a serious concern of STI is that boosts of viral replication could increase the possibility of onset of resistant mutants. We hypothesized, however, that the autologous virus rebounding during therapy interruption could enhance immune responses, thus contributing to the control of virus after treatment withdrawal. Here we describe the first prospective study that provides evidence that STI might be a feasible strategy to boost immune control of HIV.

Diagnostic Assay

Blood and Isolation of PBMC. Heparinized whole blood was diluted 1:2 in RPMI 1640 media and overlaid on Ficoll-Paque (Pharmacia Biotech, Sweden) and centrifuged. Peripheral blood mononuclear cells (PBMC) were then collected from the medium/ficoll interface and washed three times with RPMI1640 medium, resuspended in complete RPMI1640 (RPMI1640 supplemented with 10% fetal calf serum, 2 mM glutamine, 100 u/ml penicillin, 100 ug/ml streptomycin, and 50 uM 2-mercaptoethanol). Note, that the same assay can be performed in whole heparinized blood (or blood in the presence of any anticoagulants) without Ficoll separation.

Quantifying plasma HIV-1 RNA. Levels of plasma HIV-1 RNA were quantified with a RT-PCR assay (Laboratory Corporation of America, Research Triangle Park, N.C.) with a lower limit of detection of 50 RNA copies/ml.

Antigen. HIV antigens must be used as an analytical reagent for the detection of HIV-specific T cell responses. Suitable antigens include but are not limited to heat-inactivated HIV, Zinc-depleted HIV, replication-defective HIV, Gag protein, Env protein, Pol protein, Tat protein, Nef protein, Rev protein, Integrase Protein, Vpr protein, Vpu protein. In the present series of experiments we have used either heat-inactivated HIV-1 or Zinc-depleted HIV-1 vital preparations for testing in humans. Purified heat-inactivated HIV-1 virus was provided by Advanced Biotechnologies Incorporated, USA.

For monkeys, we have used Zinc-inactivated SIV preparations to detect SIV-specific immune response. To detect other virus specific immune responses, the antigen can be heat inactivated or defective virus or protein derived from the virus. To detect immune responses against tumors the antigen must be the tumor antigen or an antigen closely associated with the tumor.

Antibodies. Anti-human IL-10 neutralizing antibody was purchased from R & D Systems Inc.; PE-labelled anti-human IL-10 antibody was obtained from Biosource; PE-labelled anti-human IFN-gamma, FITC and ECD-labelled anti-human CD8, ECD and PC5-labelled anti-human CD3, FITC-labelled anti-human CD45RO, were bought from Immunotech (Marseille Cedex, France); Tri-color-labelled anti-human CD45RA and CD4 were bought from Caltag (Burlingame, Calif.).

CTL assay. Dendritic cells were generated from adherent monocytes and cultured with IL-4 and GM-CSF for 6 days as previously described (Sallusto+Lanzavecchia), then pulsed with 10 ug/ml highly purified heat-inactivated HIV antigen or with the same amount of control antigen (lysosyme) for 12 hours. Subsequently, cells were incubated with ⁵¹Cr (NEN, Boston, Mass.) for one hour, washed three time with PBS, and co-cultured with fresh autologous PBMC at various effector to target ratio for 6 hours. Supernatants were collected for measurement of ⁵Cr release. Specific release in percentage was calculated as following:

$$Specific release = \frac{Experimental release - spontaneous}{Maximum release - spontaneous} \times 100 \%$$

Activation of HIV-specific T-cells in vitro. PBMC were plated into flat-bottom 96-microtiter plates (Costar, N.Y.) at 1 million cells/well in 200 ul complete RPMI 1640 medium containing 10 ug antigen and 10 lU/well recombinant human IL-2 (rhIL-2). (Increasing the concentration of IL-2 (up to 500 U/ml) is OK, especially less antigen gives same results when IFN-gamma assayed.) Control PBMC are plated in medium containing 10 IU/well rhIL-2 without antigen. Cells are then cultured for 18 hours. Brefeldin A (Sigma, USA) is added at 2 ug/ml and the cells are incubated for another 3 hours and then collected for intracellular staining.

IFN-gamma assay. Cells were collected and aliquoted to 0.5 million cells for FACS assay. After washing twice with 1 ml PBS containing 1% BSA, cells were resuspended in 40 ul PBS/1% BSA and stained with CD8, CD4, CD3, and CD45RO antibodies for 30 minutes on ice. After washing, cells were fixed and permeabilized with 0.5 ml Fix/Per solution (containing 4% paraformaldehyde and 0.2% saponin in PBS, pH7.4) for 15 minutes on ice. Then cells were washed once with 1×per solution (0.1% saponin in PBS/1% BSA), then resuspended in 40 ul 2×Per buffer solution (0.2% saponin in PBS with 1% BSA) and incubated with IFN-gamma antibody on ice for 30 minutes. After intracellular staining cells were washed twice with 1 ml 1×Per buffer, resuspended in 0.5 ml 1% paraformaldehyde PBS buffer and analyzed on FACS (Coulter). A total of 50,000 events were acquired in every analysis.

Other than flow cytometric (FACS) analysis can also used to determine IFN-gamma or IL-10 response. This includes but not limited to ELISA and ELISPOT assays.

Interruption of IL-10 function. A neutralizing antibody against IL-10 is used to test the influence of IL-10 on IFN-gamma. All conditions are the same as described above with addition of IL-10 neutralizing antibody at 50-100 ug/ml in both control and experimental wells.

Patients

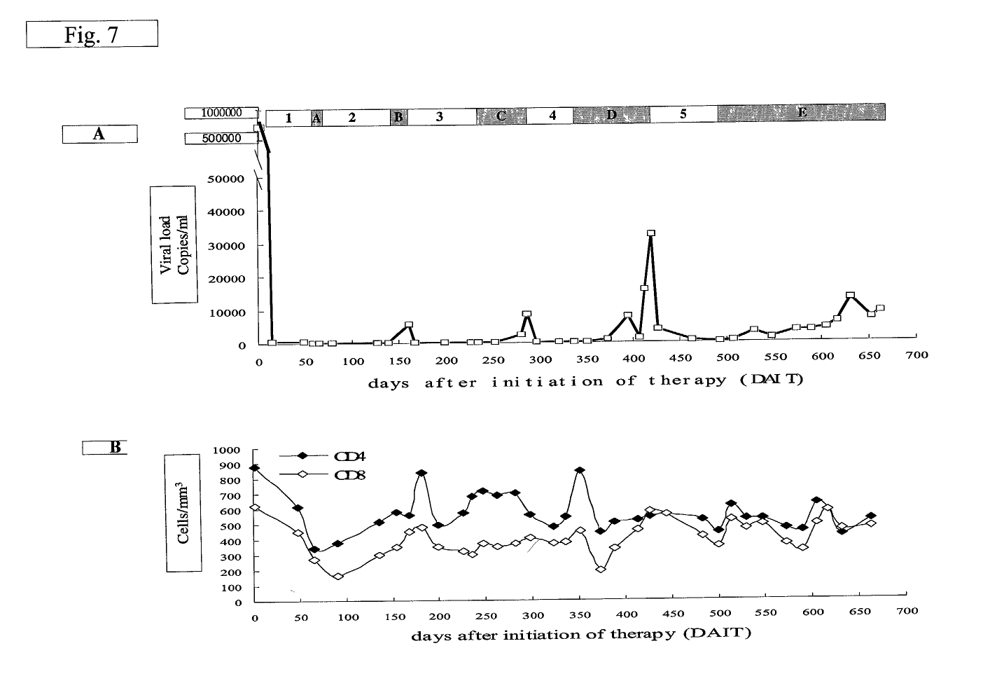

A patient from Washington, DC (the Washington patient), age 42, presented with a documented history of HIV infection was willing to undergo structured treatment interruptions. At the time the patient was enrolled in the study he did not have signs and/or clinical symptoms typical of primary HIV infection. The patient was then treated with d4T 2×40 mg, 3TC 2×150 mg and Nelf 3×750 mg, 49 days later, the drug regimen was switched to hydroxyurea-contained HAART, including d4T 2×40 mg, ddl 2×200 mg, HU 2×500 mg and Nelf 3×750 mg. Treatment was interrupted and restarted 5 times during the 95 weeks follow-up. Therapy was always different HU-containing HAART, because the patient experienced severe peripheral neuropathy. Other drug combination were (see FIG. 7): Therapy 2: d4T 2×40 mg, ddl 2×200 mg, HU 2×500 mg and Nelf 3×750 mg; Therapy 3: d4T 2×40 mg, 3TC 2×150 mg, HU 2×500 mg and Nevirapine 2×200 mg; Therapy 4: AZT 3×100 mg, 3TC 2×150 mg, HU 2×500 mg and Nevirapine 2×200 mg, Therapy 5: same as therapy 4.

Cell-mediated immune responses of five HIV seronegative individuals (negative controls, NC) and one seropositive individual (positive control, PC) have been also investigated. PC had spontaneously controlled virus replication in the absence of drug treatment. This patient's viral load has been undetectable despite an HIV-1 infection confirmed by both ELISA and Western blot.

Structured Treatment Interruption (STI)

After the Washington Patient started HAART viremia decreased to undetectable (<50 copies/ml) in 56 days of treatment (FIG. 7). In order to mimic the case of the Berlin patient, the therapy was discontinued for one week. We were concerned that a high level of virus replication would be induced by therapy interruption, but the viral load did not rebound during this time. The patient was subsequently treated for 74 days and viremia became and continued to be undetectable. In order to allow the immune system to encounter HIV antigen, therapy was scheduled to be re-started only when viremia rebounded above 5,000 copies/ml in the following STI (Interruptions B, C, D, E). Patient viremia was monitored weekly during suspension of therapy. Viremia rebounded above 5,000 copies/ml in 18 days during this second interruption. Therapy was restarted and virus was still sensitive to the treatment. Viremia declined below 50 copies/ml in 42 days. During this 3rd period of treatment viral load remained undetectable for another 33 days. During the third interruption, HIV rebounded above the threshold in 53 days. The treatment was then resumed for 50 days followed by a fourth interruption. Similar to the previous interruptions, viremia rebounded >5,000 copies/ml in 52 days, however, in this occasion the treatment was not resumed immediately. Interestingly, after a peak viremia of 9,956 copies/ml, viral load spontaneously decreased to 1121 copies/ml during the following 19 days. Unfortunately, viremia increased again to 32,161 copies/ml, and treatment was resumed for another 72 days. During the fifth STI, however, the time to rebound increased to 124 days. Subsequently, viremia plateaued at 8,000 copies/ml for another 48. Therapy was eventually restarted (FIG. 7A). Overall, the Washington patient has taken antiretroviral drugs for only 50% of his treatment period (337 of 665 days).

The CD4 and CD8 counts fluctuated during the follow up (FIG. 7B). Interestingly, controlled HIV rebound was not associated with a fall of CD4 count during this STI (FIG. 7B). CD4 count was consistently maintained above 400, and CD4/CD8 ratio mostly remained >1.

HIV-1-Specific T Cell Responses During STI

To elucidate the reasons accounting for the increased containment of the virus during STI, HIV-specific CD3+ T lymphocyte responses were measured by a sensitive and quantitative multiparameter flow cytometric assay. Intracellular IFN-gamma is considered an early response marker and produced immediately after antigen specific T cell activation in CTL and Th1 type of responses[Murali-Krishna K, Altman J, Ahmed R, et al. Counting antigen-specific CD8 T cells: A reevaluation of bystander activation during viral infection. Immunity 1998, 8: 177-87; Pitcher C, Quittner C, Picker L, et al. HIV-1-specific CD4+ T cells are detectable in most individuals with active HIV-1 infection, but decline with prolonged viral suppression. Nat. Med. 1999, 5: 518-25]. As expected, less than 0.01% HIV-specific T lymphocytes were found in the seronegative individuals. FIG. 8a represents the T cell responses of one of the five NC. In contrast to NC, 2.70% CD3+ T of the total PBMC (corresponding to 4.2% of the CD3+ T cells)(FIG. 8d) were found to be HIV-specific in the PC patient who was able to control HIV in the absence of drugs. These responses represent a pure HIV-specific T cell response, because in the absence of antigen or after stimulation with a control antigen only a very low background (<0.05%) IFN-gamma producing T cells were found (FIG. 8b).

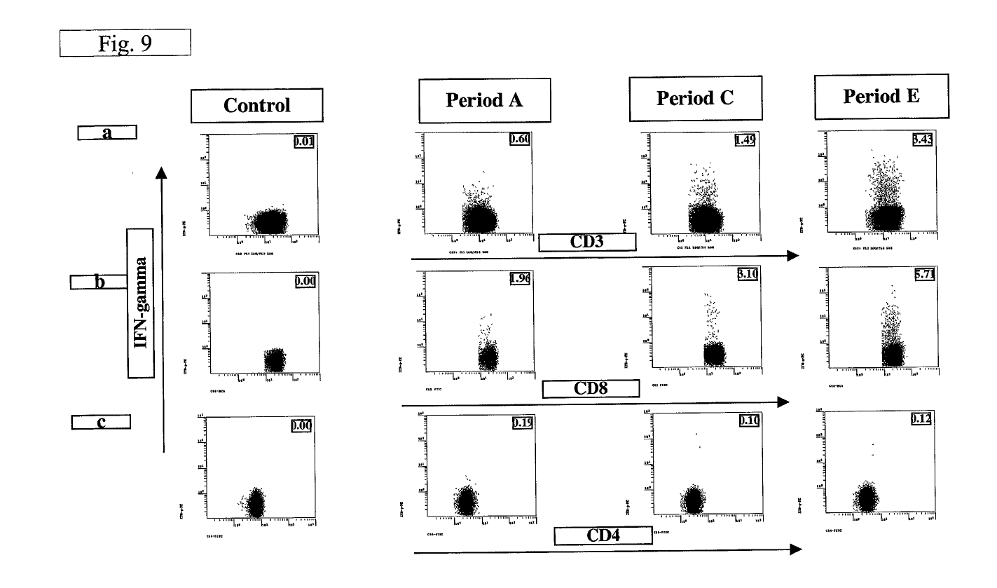

Based on these results, we quantitatively determined the evolution of T cell responses during the STI of the Washington patient (FIG. 9). This patient had a substantial amount of HIV-specific T cell response already during the first treatment interruption (0.6% of the total CD3+ T lymphocytes). This finding is consistent with previous results showing that HIV specific T cell responses are notcompletely lost if the patient is treated during primary infection. The percentage of these HIV specific T lymphocytes substantially increased (from 0.6% to 3.4%) between the first and last interruptions during STI (FIGS. 9a A, B, C) in the Washington patients.

HIV-Specific CD8 T Cell Responses During STI

To assess which population of T lymphocytes responsible for the increased HIV-specific T cell responses, we studied the HIV-specific CD8 T lymphocytes. Similar to the total T cells, less than 0.01% IFN-gamma positive cells were found in the NC and 2.9% of the CD3+ T cells were HIV-specific CD8+ T cell in the PC (FIGS. 8a,d). 2.9% represents a robust T cell response, because in the absence of antigen or after stimulation with a control antigen only a low background (<0.05%) IFN-gamma producing cells were found (FIG. 8).

At the first interruption, 1.9% of the CD3+CD8+ T lymphocytes of the Washington patient were already able to respond to HIV. Importantly, during STI, the percentage of HIV-specific CD8 lymphocytes (CD8+, CD3+, IFN+) increased from 1.9 to 5.7% (FIG. 9B).

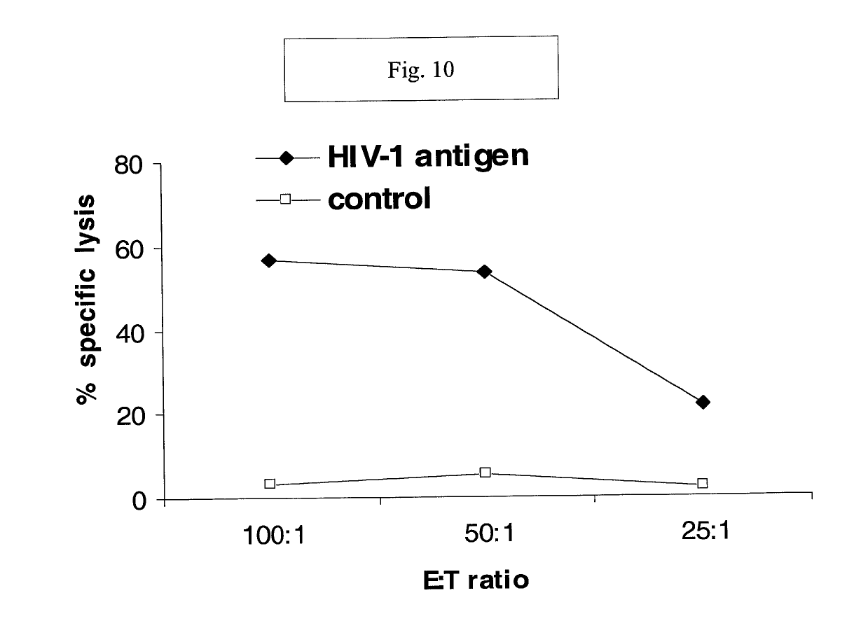

CD8+ T lymphocytes that are able to express IFN-gamma after antigen-specific stimulation have been described to correspond to antigen-specific cytotoxic T cells[Murali-Krishna K, Altman J, Ahmed R, et al. Counting antigen-specific CD8 T cells: A reevaluation of bystander activation during viral infection. Immunity 1998, 8: 177-87]. To confirm the presence of CTL responses in the Washington patient, conventional HIV specific lysis assay were performed. When HIV antigen-pulsed autologous dendritic cells were used as target cells, vigorous CTL activity (56.7%) was detected during the fifth STI (FIG. 10).

HIV-1-Specific CD4 T Helper Responses During STI.

Based ona modification of a previously described method[Pitcher C, Quittner C, Picker L, et al. HIV-1-specific CD4+ T cells are detectable in most individuals with active HIV-1 infection, but decline with prolonged viral suppression. Nat. Med. 1999, 5: 518-25] we analyzed the HIV-specific T helper lymphocytes. As expected, less than 0.01% IFN-gamma positive CD4+, CD3+ cells were found in the NC and 0.49% in the PC (FIG. 8).

In the Washington patient, HIV-specific CD4+ T helper cells were detected in all times, however, these responses did not increase during STI (FIG. 9c). Similar results were obtained when a shorter (8 hours) HIV-specific stimulation protocol was employed (data not shown).

HIV-Specific Memory T Cell Responses During STI.

CD45RA,CD45RO+ T cells have been described as memory cells. For the quantitative analysis of the HIV-specific memory lymphocytes we developed a new assay that based on stimulation of the cells with HIV antigen and stain with CD45RO antibody concomitant with CD3 and IFN-gamma antibodies.

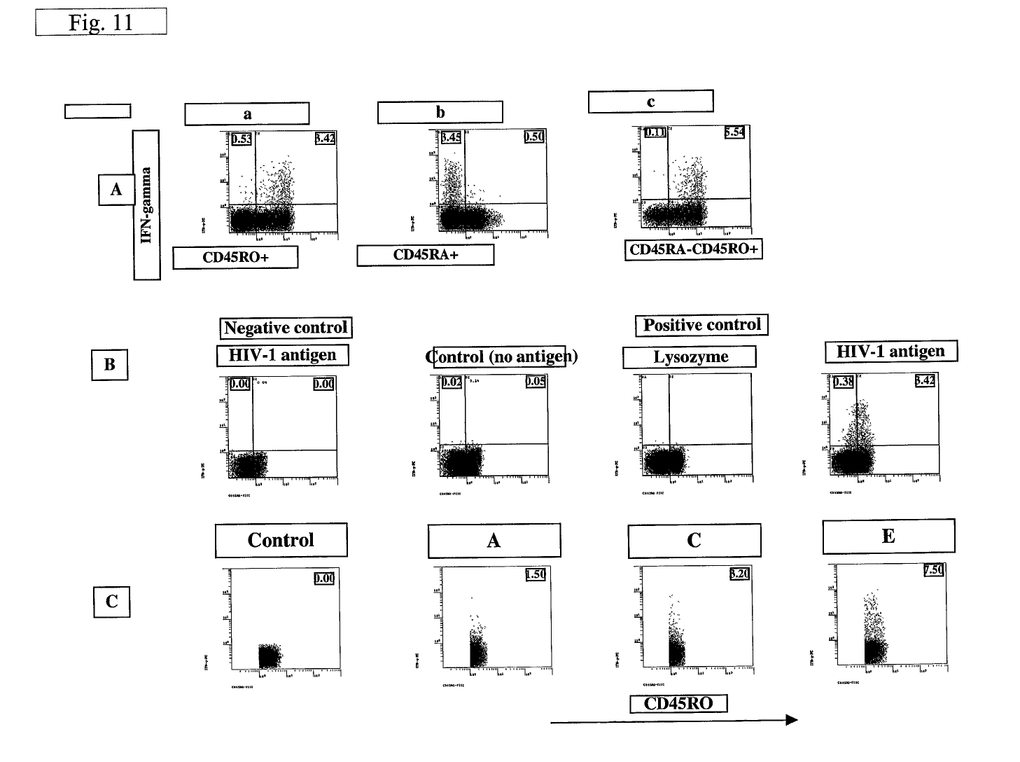

In our first experiments we have shown that the HIV-specific IFN-gamma responses derived from CD45RO+and CD45RA—were comparable (FIG. 11A). When CD45RA negative population was gated, all IFN-gamma positive T cells were CD45RO positive, suggesting that CD45RO+ T cells represents the memory cell population (CD45RA-CD45RO+). In addition, the percentage of CD45RO lymphocytes in antigen-stimulated PBMC was identical to that in unstimulated PBMC data not shown), confirming that the short HIV-specific stimulation did not allow HIV-specific memory T cells to downregulate CD45RO. Consequently, CD45RO lymphocytes that produce IFN-gamma after short antigenic stimulation represent the HIV-specific memory T cells population.

Analyzing our control patients, no CD3+CD45RO+ cells were found to produce IFN-gamma in the NC, confirming the absence of HIV-specific cells in seronegative control individuals. In contrast, 9.0% of CD3+,CD45RO+, IFN+ cells (representing 3.4% of the CD3+ T cells) were found in the the PC, indicating that high percentage of the total memory lymphocyte population was HIV-specific memory cells in the patient controlling the virus in the absence of antiretroviral therapy (FIG. 11B).

In the Washington patient, we found that HIV-specific memory lymphocytes increased from 1.5% to 7.5% between the first and the last treatment interruption during STI (FIG. 11C).

Discussion

Recently, a new technique was introduced to detect antigen-specific immune responses by in vitro stimulation with specific antigens. This is based on a sensitive and precise flow cytometeric determination of intracellular IFN-gamma expression in lymphocytes. The salient advantages of this new technique is that it is quantitative and antigen-specific T cells can be studied together with their phenotyping. We have adapted this assay to quantify HIV-specific T cell responses.

CD3+, IFN+ cells that respond to HIV represent the total population of specific T lymphocytes competent to control HIV. During the treatment of the Washington patient with STI, the percentage of HIV-specific lymphocytes increased in time. This feature distinguishes the STI of the Washington patient from HAART, because HAART is characterized by a decline of HIV-specific T-cell responses.

The CD3+,CD8+,IFN+ cells that respond to HIV-1 represent the HIV-specific CTL population. Recently, it has been demonstrated that the percentage of IFN-gamma positive CD8 lymphocytes is linearly correlated with CTL activity[Murali-Krishna K, Altman J, Ahmed R, et al. Counting antigen-specific CD8 T cells: A reevaluation of bystander activation during viral infection. Immunity 1998, 8: 177-87]. Consistent with these findings, the Washington patient had a robust CTL activity during the last treatment interruption together with a substantial IFN-gamma production in the CD8 lymphocytes.

The increased containment of viremia during sequential STI correlated with boosted CD3 and CD8 HIV-1-specific responses. This suggests that the increased suppression of HIV be likely mediated by CD3 T lymphocytes, especially by the CD8 T lymphocyte subpopulation. The finding that STI in the Washington patient can boost HIV-specific CD8 T cell responses is in sharp contrast with the results obtained with HAART, in which a decline of CTL was found after the suppression of viral replication[Pitcher C, Quittner C, Picker L, et al. HIV-1-specific CD4+ T cells are detectable in most individuals with active HIV-1 infection, but decline with prolonged viral suppression. Nat. Med. 1999, 5: 518-25; Ortiz G, Nixon D, Markowitz M, et al. HIV-1-specific immune responses in subjects who temporarily contain virus replication after discontinuation of highly active antiretroviral therapy. J. of Clinical Investigation 1999,104: R13-8].

The CD3+, CD4+, IFN+ cells that respond to HIV represent the HIV-1 specific T-helper population. These cells are found during the infection to support anti-HIV CTL activity[Pitcher C, Quittner C, Picker L, et al. HIV-1-specific CD4+ T cells are detectable in most individuals with active HIV-1 infection, but decline with prolonged viral suppression. Nat. Med. 1999, 5: 518-25]. The loss of HIV-specific T-helper cells has been associated with progressive infection. Our results confirmed the presence of CD4 T-helper responses in the Washington patient (FIG. 9c). This is in agreement with previous reports, showing HIV-specific T helper responses in patients treated early after infection. We did not find an enhancement of the HIV specific T helper response by STI, despite an increased containment of virus was observed. It has been shown that T-helper cells are not essential to maintain CD8+ T-cell responses (Di Rosa J Exp Med 1996, 183; 2153). In fact, some long-term non-progressors do not have a high percentage of CD4+ T-helper cells. The presence of CD4 T-helper cells in this patient might have been sufficient to support a vigorous CD8 mediated response. In contrast to HAART, where T-helper responses decline in time during the treatment, STI conserved these responses in the Washington patient.

CD3+,CD45RO+,IFN+ cells that respond to HIV represent the HIV-specific memory lymphocytes, that are the long-lived population of HIV-specific cells that develop as a consequence of antigenic stimulation[Kalams S, Goulder P, Walker B, et al. Levels of human immunodeficiency virus type 1-specific cytotoxic T-lymphocyte effector and memory responses decline after suppression of viremia with highly active antiretroviral therapy. J. of Virology 1999, 73: 6721-8]. We found that 9.0% of the total memory cells are HIV-specific in the patient able to control HIV in the absence of drugs (FIG. 11b, positive control). In contrast, in the Washington patient treated with STI, we observed that the HIV-specific memory cell population increased by 5 fold from 1.5% to 7.5% (FIG. 11c). These results implicate that successful auto-immunization have been achieved by STI. Since memory T cells respond to antigen challenge faster and stronger than naive T cells after reexposure to the same antigen, they are expected to initiate effective clearance of virus. We suggest that the increase of HIV-specific memory T cell population account for the prolonged control during the last treatment interruption in the Washington patient.

### Example 5

### HIV-Specific Antibody Response Induced During

During Th1-type of immune responses, effector T cells mediate a variety of functions that are the most important in antiviral immunity: (1) killing infected cells, (2) activate macrophages, allowing them to destroy intracellular microorganism, and (3) activate B cells to produce strongly opsonizing antibodies belonging to certain IgG subclasses (IgG1 and IgG3).

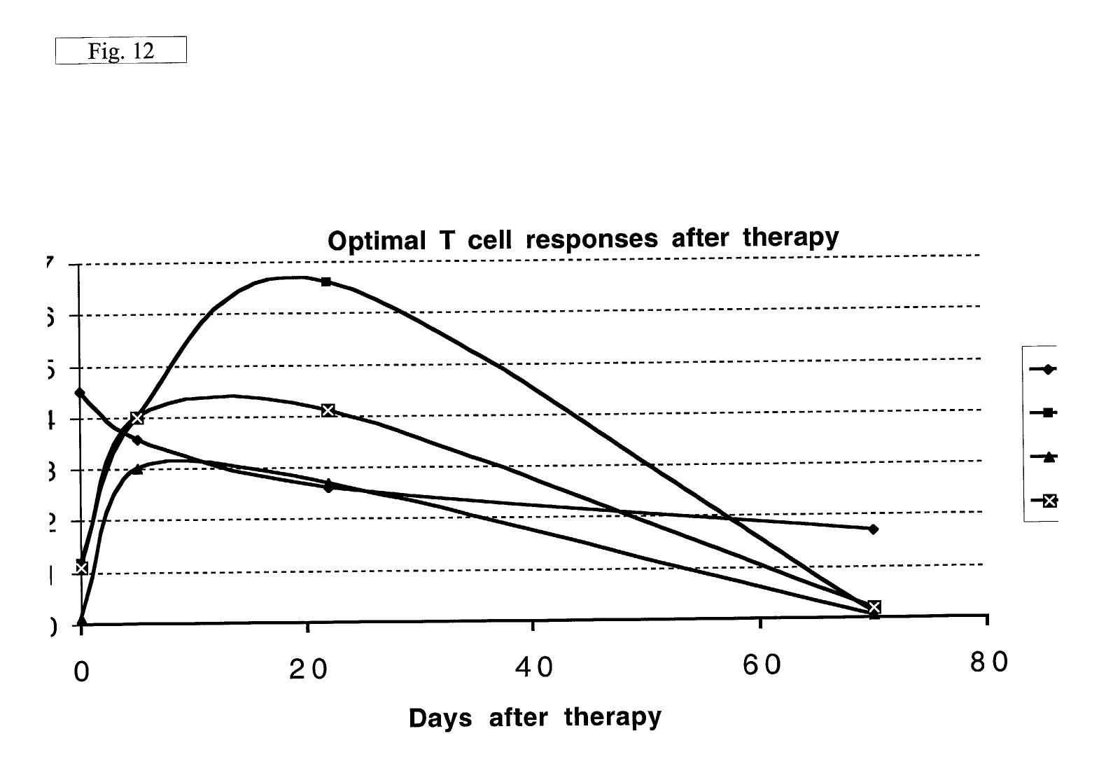

Table 3 demonstrates the neutralizing antibody response after interruption D (see FIG. 7). Although we did not subclass these antibody responses, we found that the activity measured here is corresponding with the IFN-gamma production (VIR) in the patient (FIG. 12). Therefore, the neutralizing activity might correspond to a Th1-mediated opsonizing antibody response that also can neutralize. This indicate that Th1 type of responses are induced during STI.

TABLE III
neutralizing antibodies after interruption D, therapy cycle 5 (see FIG. 7)

| | | | |
|---|---|---|---|
| | RESTING NT BL56 | | RESTING NT BL56 |
| VIRUS | J45CPpIII | VIRUS | J40GAp |
| | (CCR3/CCR5/CXCR4) | | (CCRS) |
| TClD50 | 68.3 | TClD50 | 17 |
| OD 1° dil | 951 | OD 1° dil | 1061 |
| | (min 905/max 1049) | | (min 955/max 1166) |

| Days, after | | | |
|---|---|---|---|
| therapy 5 starts | % Neutraliz. | % Neutraliz. | Viral load |
| 0 | | | |
| 1/50 | 0.00 | 0.00 | 32161 |
| 1/200 | 0.00 | 0.00 | |
| 1/800 | 0.00 | 0.00 | |
| 1/3200 | 0.00 | 3.00 | |
| 5 | | | |
| 1/50 | 97.00 | 97.80 | 3730 |
| 1/200 | 92.00 | 81.60 | |
| 1/800 | 0.00 | 60.00 | |
| 1/3200 | 0.00 | 10.50 | |
| 14 | | | |
| 1/50 | 97.00 | 96.20 | 463 |
| 1/200 | 21.00 | 82.30 | |
| 1/800 | 0.00 | 11.80 | |
| 1/3200 | 0.00 | 44.70 | |
| 22 | | | |
| 1/50 | 98.00 | 88.00 | 500 |
| 1/200 | 8.00 | 14.50 | |
| 1/800 | 0.00 | 55.90 | |
| 1/3200 | 0.00 | 13.00 | |
| 40 | | | |
| 1/50 | 98.00 | 98.00 | 185 |
| 1/200 | 6.00 | 95.00 | |
| 1/800 | 0.00 | 92.40 | |
| 1/3200 | 0.00 | 14.00 | |
| 48 | | | |
| 1/50 | 96.00 | 97.00 | 60 |
| 1/200 | 36.00 | 82.00 | |
| 1/800 | 0.00 | 3.40 | |
| 1/3200 | 0.00 | 0.00 | |
| 61 | | | |
| 1/50 | 84.00 | 74.60 | <50 |
| 1/200 | 12.00 | 19.20 | |
| 1/800 | 0.00 | 11.00 | |
| 1/3200 | 0.00 | 5.00 | |

### Example 6

### Evidence that the Optimal Therapy Period is Short

FIG. 12 illustrates the change HIV-specific T cell responses after initiation of HAART (treatment period 5) in the Washington patient as % of IFN-gamma producing cells. It shows that shortly after effective HAART was initiated, the viral load (VL log) rapidly decreased and HIV-specific T cell responses, characterized by % of HIV-specific CD3, CD4 and CD8 cells, increased to a peak value between weeks 1 and week 4, then decreased within about 80 days. This figure suggests that drug treatment should be stopped at the peak of these immune responses. Indeed, the monkey protocol of Example 3 has only 3 weeks of treatment, which is the preferred embodiment.

Not all patients are expected to be able to mount these HIV-specific immune responses after initiation of therapy. Whether the patient responds or not depends at least on his immunological status and the viral load upon initiation of treatment for a given cycle. We suggest that even if the patient does not respond to a first treatment cycle, therapy should be interrupted for a short period of time, since there is a chance that the repeated therapy interruptions may increase HIV-specific immune responses even in the late stage patients.

### Example 7

### IL-10 Production

As we already pointed out, sometimes during the STI (not always) IL-10 production has been detected. IL-10 might be produced by HIV or by other pathogen as Herpes viruses. IL-10 also inhibits IFN-gamma production therefore having a damaging effect to the HIV-specific Th1 type responses that can control viruses.

We assayed the viral load and intracellular production of both IFN-gamma and IL-10 as described in Example 4, above, in order to monitor the patient's immune system. The assay for IL-10 is the same as for IFN-gamma, except that IL-10 antibodies are used instead of IFN-gamma antibodies. Together, these diagnostic assays can predict the efficacy of the Th1 immune responses to control HIV, and may be used to predict the new setpoint of viral load after interruption oftherapy, and may predict whether the patient would benefit from immunomodulatory therapies.

Other than flow cytometric (FACS) analysis can also used to determine IFN-gamma or IL-10 response. This includes but not limited to ELISA and ELISPOT assays.

IL-10 Production and its Relationship with Viral Load

We monitored the IL-10 and IFN-gamma and viral load of Washington patient closely before and after the 5^{th} therapy cycle of his STI (FIG. 7). IL-10 was detectable during the entire course of monitoring without antigenic (HIV) restimulation in vitro. The highest IL-10 production, however was observed on day—9 before the 5^{th} therapy cycle in both CD3+(over 3%) and CD3-(over 9%) cell populations. Importantly, this high IL10 production preceded the high rebound of HIV in the absence of therapy. IL-10 production was completely inhibited 22 days after starting the 5^{th} therapy cycle.

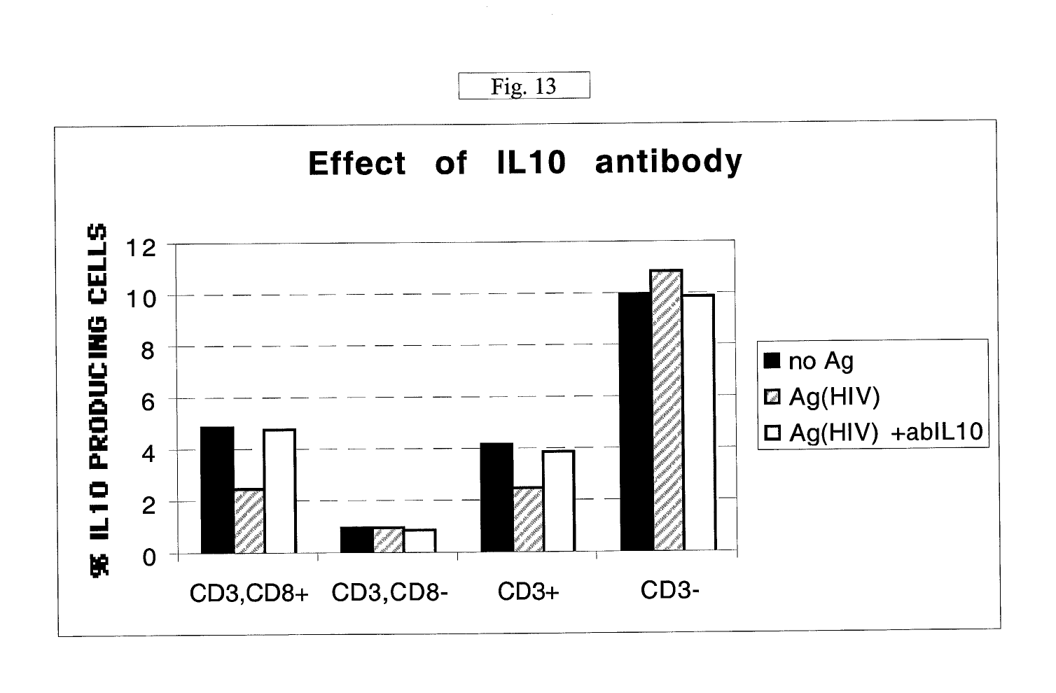

FIG. 13 shows th e effect of HIV on IL-1 production and the ability of IL-10 to reverse its effect. HIV antigen decreased IL-10 production and IL10 antibody restored the effect of HIV. The effect is only visible in the T cell (CD3+),especially in the CD8+ population. This data demonstrates that IL-10 antibody inhibits the over-activation of T cells by HIV. In the presence of IL-10 antibody, T cells do not die showing that it is IL-10 and not HIV that is the direct eliminator of these cells. Interestingly, the IL-10 neutralization antibody is able to recover IL-10 production after HIV stimulation in the same sub-population. This effect suggested that the inactivation of the IL-10-producing cells was mediated by IL-10 itself. It appears to be a negative feedback on these cells because HIV is able to further stimulate IL-10 production, and the increased IL-10 kills the IL-10-secretingcell population. Similar results were found on day 7 after stopping therapy: almost all of IL-10-producing cells in both CD3+ and CD3-had been destroyed by this time by additional HIV antigen. From these data, we concluded that HIV is using the IL-10 pathway to escape from immune control.

Similar activity is expected if IL10 is produced by other pathogens, e.g. Herpes viruses.

IL-10 production is a sign of antigenic over-activation of T cells (see general diagram: too much antigen exhausts the immune system). This might happen in the presence of too much HIV, that over-activates T cells. These over-activated T cells produce IL-10 then they die. IL-10 also inhibits Th-1 responses that fight against HIV. This is new approach, because IL-10itself has beenpreviously suggested to use as antiretroviral therapy. Here we have evidence the opposite result, that IL-10 production by the body interferes with the immune system's ability to control HIV. Therefore, instead of IL-10, we suggest that the administration of IL-10 neutralizing antibodies will be beneficial for the patients.

Inhibition of T Cell Responses by IL-10

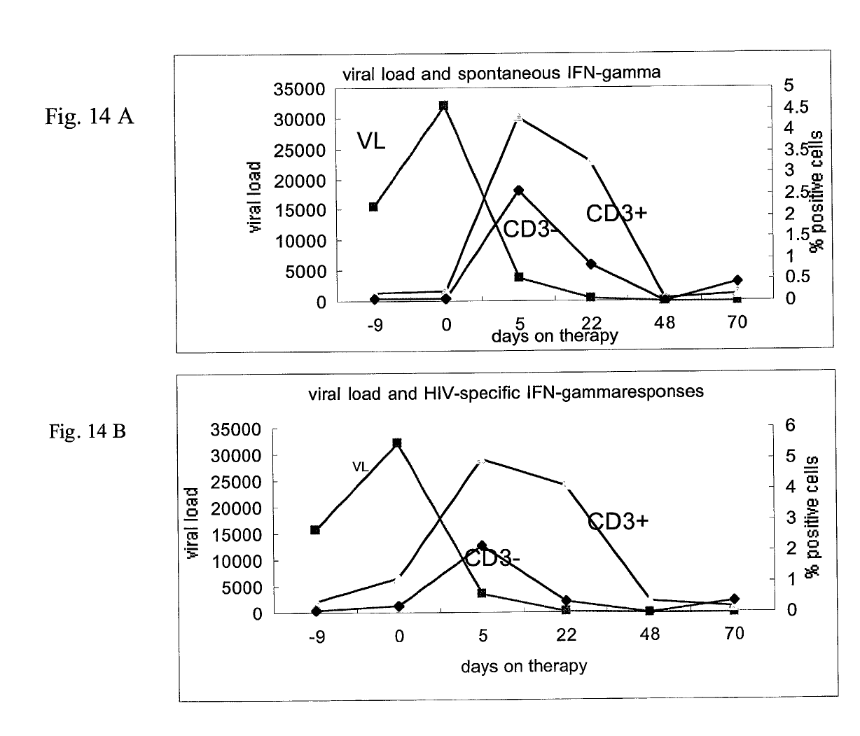

Similar amounts of spontaneous and HIV-specific T-cell responses (indicated as % of IFN-gamma-positive cells) were detectable after the 5^{th} therapy cycle (FIG. 14). This data demonstrates that in vivo HIV stimulated a maximal amount of IFN-gamma production. However, IFN-gamma production was very low 9 days before the therapy start that preceded the rebound of viral load. This low IFN-gamma production and rebound occurred in connection with high IL-10 production.

Because IL-10 expression in vivo in the absence of therapy impaired the T cell responses, we designed an experiment in vitro to interrupt the function of IL-10. When various amounts of an IL-10 neutralizing antibody were added into our system, T-cell responses (including both CD4 and CD8) were dramatically increased and correlated with the amount of IL-10 antibody applied.

An exception occurred on day 5 after drug therapy began when IFN-gamma responses peaked (Table IV). Here the IL-10 neutralizing antibody was not able to further increase IFN-gamma responses, which suggested that the impairment of T cells responses induced by IL-10 was overcome by strong T cell responses (measured by IFN-gamma). This suggests that IL-10 cannot inhibit any more strong Th1 responses and the immune system is polarized to control HIV. It also suggests that after strong Th1 responses have been induced, control of HIV can be achieved in the absence of other immunomodulatory therapies.

TABLE IV
Maximal amount of T cell responses

| | | | Stimulated | | Stimulated with | | | |
|---|---|---|---|---|---|---|---|---|
| | Unstimulated | | with HIV | | HIV + Ab IL10 | | PMA + A23187 | |
| | IFN-g | IL-10 | IFN-g | IL-10 | IFN-g | IL-10 | IFN-g | IL-10 |
| CD3+ | 4.30% | 4.00% | 5.00% | 5.10% | 4.80% | 3.30% | 9.50% | 2.20% |
| CD3− | 2.60% | 1.90% | 2.20% | 2.10% | 2.40% | 1.90% | 3.60% | 1.30% |
| CD3+ CD8+ | 2.90% | 3.30% | 4.00% | 3.80% | 3.60% | 3.00% | 3.90% | 1.00% |
| CD3+ CD8− | 2.60% | 2.00% | 3.00% | 3.70% | 2.80% | 1.70% | 3.00% | 0.50% |

PMPA+A23187 stimulation is an unspecific activation, represents the maximum percentage of IFN-gamma expressing cells after activation. Table IV shows that HIV-specific CD8+ and CD4+ cells (measured as CD3+CD8−) are the same amount that after maximum stimulation. This suggest that at this point IFN-gamma responses have been maximized in this patient, and that there aren't any more cells that can be activated at this point of time to make IFN-gamma.

The inhibition of Th1 responses was also manifested through comparison between IL-10 and IFN-gamma production in vivo. When IL-10 decreased on day 0, IFN-gamma increased immediately. When IL-10 increased on day -9, a reduction of IFN-gamma responses occurred. Later, due to the effect from HIV antigen stimulation and drug therapy, the relationship between IFN-gamma and IL-10 was not as clear as before day 0. Therefore besides viral load, we suggest that host immune responses should be taken into consideration as a parameter for stopping drug therapy. Specifically, IFN-gamma production needs to be higher than IL-10 production. This can be assayed as diagnostic parameter.

It has been difficult to explain why we and other investigators find HIV-specific T-cells responses in patients not treated with antiretroviral therapy. These responses are high early after infection and decline in time. These responses are not detectable in AIDS. This could be explained by the IL-10 and IFN-gamma equilibrium in chronically infected patients. HIV induces both IL-10 and IFN-gamma responses. If IL-10 production is high, IFN-gamma production is low and vice versa. IL-10 producing cells are short lived. When they die, IFN gamma producing cells will try to control HIV and kill some infected cells. If this is not successful, the number of IL-10 producing cells rises again and the number of IFN-gamma producing cells decreases.

### Example 8

### Data Supporting the use of Th1 Activating and Th2 Inhibiting Adjuvants in STI

As we described above, various modes of antiretroviral treatment, including STI, can be enhanced with Th1 inducing or Th2 inhibiting agents. The question is now what to use to achieve an optimal Th1 response to achieve control of HIV. Here we provide an example for Th1 activation (see IL-2) and Th2 inhibition (see IL-10 antibodies) and compare the use of IL2 and IL-10 antibodies.

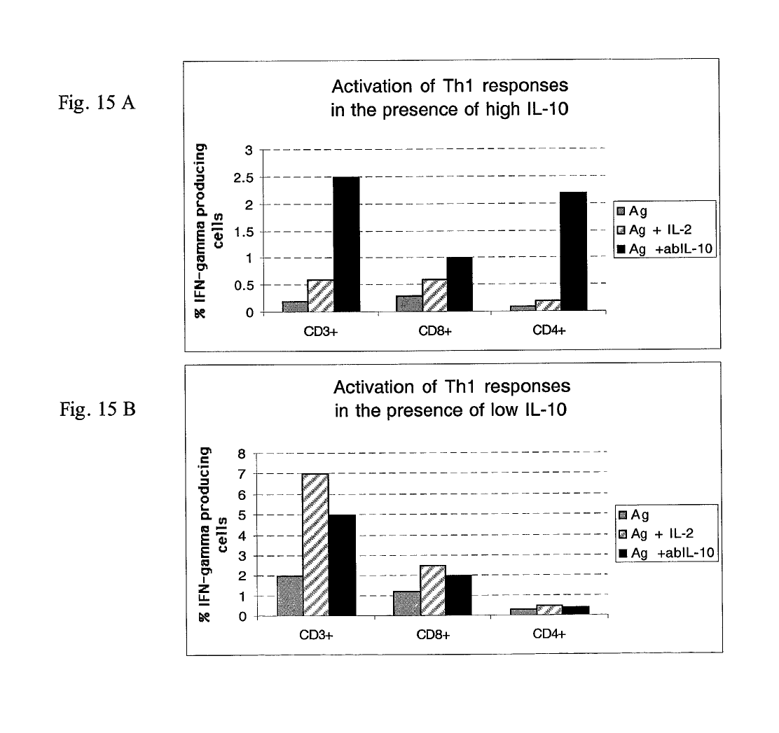

FIG. 15 shows that IL-2 can induce Th1 responses, therefore we suggest the use of IL-2 in STI. Especially, in the preferred embodiment IL-2 therapy is very efficient when IL-10 is low and IFN-gamma production is already higher (FIG. 15, upper panel). This might happen in a patient treated early after infection or after several STI. With our diagnostic assay the patient can be monitored. Those who can produce an effective IFN-gamma response (more than 1% HIV-specific IFN-gamma producing T cells) after 1-6 weeks (preferred 3 weeks) drug treatment therapy in a given STI cycle might have their immune responses boosted with one or more doses of IL-2 shortly before or after drug treatment is stopped.

IL-2 therapy has also been found to be efficient if IFN-responses are there but they are inhibited by IL-10 (FIG. 15, lower panel). In this case IL-2 doubled CD3+ T cells responses, including CD8 and CD8 responses. This is also a very significant increase.

In general, we have shown that IL-2 treatment significantly increases HIV-specific Th1 responses, independently from the amount of IL-10. It is preferred to administer IL-2 when viral load is low. Therefore, IL-2 therapy is preferred during the treatment phase of STI and during the STOP phase when the viral load is low. IL-2 therapy is most preferred when it is likely to maximize Th 1 responses during the time frame when Th1 responses are already somewhat enhanced, that is, during the therapy phase and more particularly, toward the end of the therapy phase and at the beginning of the STOP phase when these phases are set to coincide with enhanced Th1 responses.

FIG. 15 also shows the comparison between IL-2 and IL-10 neutralizing antibodies. IL-10 antibody treatment was also very effective to induce HIV-specific Th1 responses. Administration of IL-10 antibody increased IFN-gamma production in T cells more than 10 times, especially when IL-10 production was high. In this case, it had an especially a strong activating effect on CD4 cells, a cell population that IL-2 does not activate so efficiently. Therefore, IL-10 antibody treatment is preferred for patients that have high percentages of IL-10 producing cells. Administration of the IL-10 antibody may also benefit patients with low levels of IL-10 producing cells, however to a lesser extent than IL-2. The IL-10 antibody might be used weekly during STI, especially if the patient is co-infected with other pathogens inducing production of IL-10. Alternatively, the IL-10 antibody should be administered if the patient's viral load rebounds and HIV stimulates IL-10 production.

Based on this data, we also think that co-administration of IL-10 antibodies and IL-1 may provide further benefit to patients on STI. Indeed, generally it should be noted that Th1 inducing therapies combined with Th2 inhibiting therapies would result in the most optimal stimulation of Th1-mediated immmune responses and control of HIV. Which therapy in which combination might be decided based on tolerability, safety and efficacy. One strong candidate is for this effect is a molecule, called CpG sequences [SmithKline Beecham] that can do both: activate Th1 and inhibit Th2 responses.

What is claimed is:

1. A method of autovaccination against a pathogen present in the body using an optimum dose of the pathogen itself as an antigen to increase pathogen-specific immune responses.

2. The method of claim 1, wherein the regulation of the dose of the antigen is achieved by drug therapy able to inhibit the amount of the pathogen in the body.

3. The method of claim 1, wherein pathogen-specific immune responses control the pathogen after cessation of drug therapy.

4. The method of claim 1, whereby the patient is exposed to a dose of pathogen insufficient to exhaust the patient's antigen-specific T cell responses.

5. The method of claim 1, wherein the pathogen is selected from the group consisting of viruses, intracellular parasites, and tumors.

6. The method of claim 1, wherein the pathogen is a human immunodeficiency virus.

7. The method of claim 1, wherein the pathogen-specific immune responses are T cell mediated immune responses.

8. The method of claim 7 wherein the pathogen-specific immune responses are HIV-specific T cell responses.

9. The method of claim 8 wherein the HIV-specific T cell responses are an increase in the percentage of HIV-specific CD4+ cells.

10. The method of claim 9 wherein HIV-specific CD4+ cells are assayed as HIV antigen-induced IFN-gamma producing CD4+ T cells.

11. The method of claim 9 wherein the HIV-specific CD4+ cells represent the percentage of HIV-specific T-helper type 1 cells in the patient.

12. The method of claim 8 wherein the HIV-specific T cell responses are an increase in the percentage of HIV-specific CD8+ cells.

13. The method of claim 12 wherein HIV-specific CD8+ cells are assayed as HIV antigen-induced IFN-gamma producing CD8+ T cells.

14. The method of claim 12 wherein the HIV-specific CD8+ cells represent the percentage of HIV-specific cytotoxic T cells in the patient.

15. The method of claim 8 wherein the HIV-specific T cell responses are an increase in the percentage of HIV-specific CD3+ cells.

16. The method of claim 15 wherein HIV-specific CD3+ cells are assayed as HIV antigen-induced IFN-gamma producing CD3+ T cells.

17. The method of claim 15 wherein the HIV-specific CD3+ cells represent the percentage of total HIV-specific T cells in the patient.

18. The method of claim 8 wherein the HIV-specific T cell responses are an increase in the percentage of HIV-specific memory cells.

19. The method of claim 18 wherein the percentage of HIV-specific memory cells is assayed as HIV antigen-induced IFN-gamma producing CD45RO+, CD3+ cells.

20. The method of claim 18 wherein the HIV-specific memory cells represent the percentage of HIV-specific peripheral memory T cells in the patient.

21. The method of claim 1 wherein the pathogen-specific immune responses are an increase in the percentage of HIV-specific CD3− cells.

22. The method of claim 21 wherein HIV-specific CD3- cells are assayed as HIV antigen-induced IFN-gamma producing CD3− cells.

23. The method of claim 21 wherein the HIV-specific CD3− cells represent the percentage of non-lymphocytic HIV-specific cells in the patient.

24. The method of claim 1, wherein the increase in pathogen-specific immune responses is achieved with a combination of drugs and one or more immunoregulatory adjuvants.

25. The method of claim 24, wherein the adjuvants are either stimulators of Th1 responses or inhibitors of Th2 responses or the mixtures of thereof.

26. The method of claim 25, wherein the stimulators of Th1 responses are cytokines.

27. The method of claim 26, wherein the cytokine is selected from the group consisting of IL-12, IL-2, Retinoids, IL-18, IFNγ, Interferon α, Ribavirin, and Fludarabin, CpG and mixtures thereof.

28. The method of claim 25, wherein the stimulators of Th2 responses are cytokines.

29. The method of claim 28, wherein the cytokine is selected from the group consisting of antibodies against IL-10, antibodies against IL-4, antibodies against IL-5, SB 203580, suplatast tosilate, suramin, Teophillin, corticosteriods, CpG, and mixtures thereof.

30. A method of autovaccination against a pathogen present in the body using an optimum dose of the pathogen itself as an antigen to increase pathogen-specific immune responses wherein autovaccination is achieved by intermittent administration of a drug therapy.

31. The method of claim 30, wherein the drug therapy is highly active antiretroviral therapy.

32. The method of claim 31, wherein the drug therapy is capable of reducing the viral load of the body to 200-500 copies per ml plasma within two to four weeks.

33. The method of claim 32, wherein the drug therapy is a) AZT, 3TC and a protease inhibitor, b) hydroxyurea, one or more reverse transcriptase inhibitors and one or more protease inhibitors.

34. The method of claim 33, wherein the reverse transcriptase inhibitor is selected from ddl, d4T, 3TC, AZT, delaviridine, abacavir, adefovir, nevirapine, efavirenz, lubocavir, and mixtures thereof.

35. The method of claim 33, wherein the protease inhibitor is selected from indinavir, saquinavir, ritonavir, nelfinavir, GW 141, and mixtures thereof.

36. The method of claim 33, wherein the drug therapy is a hydroxyurea, ddl and d4T.

37. The method of claim 15, wherein the drug therapy includes hydroxyurea and ddl.

38. A method of autovaccination against a pathogen present in the body using an optimum dose of the pathogen itself as an antigen to increase pathogen-specific immune responses wherein autovaccination is achieved by administration of a suboptimal drug therapy that does not completely inhibit the amount of the pathogen.

39. The method of claim 38, wherein autovaccination of HIV is achieved by combination of didenosine and hydroxyurea treatment.

40. The method of claim 39, wherein the optimum dose of HIV is above 200-500 copies/ml and below 10,000 copies/ml.

41. The method of claim 39, wherein optimum dose of HIV is maintained for longer than one year.

42. The method of claim 38, wherein the treatment allows interruptions of drug intake.

43. The method of claim 42, wherein the treatment allows drug holidays of more than one week's duration.

44. The method of claim 42, wherein the treatment allows holidays of up to eight weeks duration.

45. A method of autovaccination against a pathogen present in the body using an optimum dose of the pathogen itself as an antigen to increase pathogen-specific immune responses using an intermittent drug therapy, wherein the steps of the intermittent drug therapy comprise administering a drug therapy in cycles for inhibiting the pathogen; each cycle having a treatment phase and an interruption phase; wherein at least one of the cycles has a treatment phase followed by an interruption phase that ends upon relapse; followed by a cycle having a treatment phase that lasts until pathogen-specific immune responses develop.

46. The method of claim 45, wherein the treatment phase has a period of about 1-6 weeks.

47. The method of claim 46, wherein the treatment phase has a period of about 3 weeks.

48. The method of claim 45 comprising more than one cycle.

49. The method of claim 48 comprising five cycles.

50. The method of claim 48 comprising from five cycles until the end of the patient's life.

51. The method of claim 45, wherein relapse is defined as an increase in viral load in the plasma to about 2,000 copies/ml or more.

52. The method of claim 45, wherein relapse is defined as an increase in viral load in the plasma to about 5,000 copies/ml or more.

53. The method of claim 45, wherein relapse is defined as an increase in the viral load in the plasma is between 5,000 to 50,000 copies/ml.

54. The method of claim 45, wherein relapse is defined as an increase in the viral load in the plasma is between 10,000 to 100,000 copies/ml.

55. The method of claim 45, further comprising the step of administering one or more immunoregulatory adjuvants.

56. The method of claim 55, wherein the adjuvants are either stimulators of Th1 responses or inhibitors of Th2 responses or the mixtures of thereof.

57.The method of claim 56, wherein the stimulators of Th1 responses are cytokines.

58. The method of claim 57, wherein the cytokine is selected from the group consisting of IL-12, IL-2, Retinoids, IL-18, IFNγ, Interferon α, Ribavirin, and Fludarabin, CpG and mixtures thereof.

59. The method of claim 56, wherein the stimulators of Th2 responses are cytokines.

60. The method of claim 59, wherein the cytokine is selected from the group consisting of antibodies against IL-10, antibodies against IL-4, antibodies against IL-5, SB 203580, suplatast tosilate, suramin, Teophillin, corticosteriods, CpG, and mixtures thereof.

61. The method of claim 55, wherein the one or more adjuvants are administered when the pathogen-specific immune responses are maximized.

62. The method of claim 55, wherein the one or more adjuvants are administered after the pathogen-specific immune responses have been maximized.

63. A method of measuring an immune system's competence against a pathogen, the steps comprising measuring changes in the pathogen-specific immune responses and pathogen load.

64. The method of claim 63, wherein the pathogen-specific immune responses are HIV-specific T cell responses.

65. The method of claim 63, further comprising of measuring the HIV-specific IFN-gamma responses at the beginning of and during drug treatment to determine the magnitude of the immune responses.

66. The method of claim 64 further comprising the steps of measuring the viral load in the plasma and HIV-specific IFN-gamma responses at the beginning and during the treatment to determine the efficacy of autovaccination.

67. The method of claim 63 further comprising the step of measuring IL-10 production.

68. The method of claim 67, measuring IL-10 production to determine which immunomodulatory adjuvants to use in combination with autovaccination.

69. The method of claim 64 further comprising the steps of measuring IFN-gamma and IL-10 during a treatment phase, and terminating the treatment phase at the point when the viral load reached less than 500 copies/ml, HIV-specific IFN-gamma+, CD3+ cells are greater than 2% and if the ratio of the percentage of cells producing IFN-gamma to the percentage of cells producing IL-10 is greater than 10, thereby ending the treatment phase.

70. A diagnostic test for immune system competence against Human Immunodeficiency Virus, comprising the steps of testing the viral load in the plasma and the HIV-specific interferon-gamma production in different cell types.

71. The diagnostic test of claim 70, wherein the cells are activated by a replication incompetent virus.

72. The diagnostic test of claim 70, wherein the cells are activated by a heat inactivated virus.

73. The diagnostic test of claim 70, wherein the cells are activated by a zinc inactivated virus.

74. The diagnostic test of claim 70, wherein the cells are activated by a replication competent virus.

75. The diagnostic test of claim 70, wherein the HIV-specific interferon-gamma production is tested in CD3−, CD3+, CD4+, CD8+ and CD45RO+ cells.

76. The diagnostic test of claim 75, wherein HIV-specific IFN gamma production is tested in CD4+ cells measured as the percentage of CD3+ and CD8− cells.

77. The diagnostic test of claim 75, wherein HIV-specific IFN gamma production is tested in CD8+ cells measured as the percentage of CD3+ and CD8+ cells.

78. The diagnostic test of claim 75, wherein HIV-specific IFN gamma production is tested in CD45RO memory cells measured as the percentage of CD3+ and CD45RO cells.

79. The diagnostic test of claim 70, wherein the test is carried out before starting therapy and when the viral load reaches <500 copies/ml in the plasma.

80. The diagnostic test of claim 79, wherein the test is carried out before starting therapy and 2-6 weeks later.

81. The diagnostic test of claim 70, wherein low viral load and high percentabe of interferon-gamma producing cells demonstrates the competence of the immune system against Human Immunodeficiency Virus.

82. The diagnostic test of claim 70, wherein low viral load and high percentage of interferon-gamma producing cells suggests that the viral load will be controlled after interruption of therapy.
